(19) United States
(12) Patent Application Publication
(10) Pub. No.: US 2023/0017661 A1
(43) Pub. Date: Jan. 19, 2023

(54) COMPOSITIONS AND MODULAR NANO- AND MICROPARTICLES FOR THE DELIVERY OF VARIOUS AGENTS AND USE THEREOF

(71) Applicant: Veri Nano Inc., Cambridge, MA (US)

(72) Inventors: Guillermo U. RUIZ ESPARZA HERRERA, Boston, MA (US); Kanny CHANG, Brighton, MA (US); Ketian ZHANG, Boston, MA (US); Samson AFEWERKI, Uppsala (SE)

(73) Assignee: Veri Nano Inc., Cambridge, MA (US)

(21) Appl. No.: 17/784,469

(22) PCT Filed: Dec. 11, 2020
(86) PCT No.: PCT/US2020/064565

§ 371 Date: Jun. 10, 2022

Related U.S. Application Data
(60) Provisional application No. 62/948,039, filed on Dec. 13, 2019.

(51) Int. Cl. C08B 37/16; A61K 31/07; A61K 31/436; A61K 45/06; C07H 15/00

(52) U.S. Cl. CPC C08B 37/0015; A61K 31/07; A61K 31/436; A61K 45/06; C07H 15/00; A61K 38/00

(57) ABSTRACT

The present invention relates to a cyclic polysaccharide compound and particle comprising a plurality of said cyclic polysaccharide compounds and one or more agent, wherein the plurality of cyclic polysaccharide compounds form a hollow sphere, and the one or more agent is encapsulated within the hollow sphere; and wherein the one or more agent is non-covalently associated with the cyclic polysaccharide compound, optionally further comprising a surfactant. The particles may comprise one or more additional drug. The invention further relates to pharmaceutical, cosmetic, or nutraceutical use of the particles.

### FIELD OF THE INVENTION

The present invention relates to a cyclic polysaccharide compound and particle comprising a plurality of said cyclic polysaccharide compounds and one or more agent, wherein the plurality of cyclic polysaccharide compounds form a hollow sphere, and the one or more agent is encapsulated within the hollow sphere, optionally further comprising a surfactant. The particles may comprise one or more additional drug. The invention further relates to pharmaceutical, cosmetic, or nutraceutical use of the particles.

### BACKGROUND OF THE INVENTION

Modular nano- and microparticles are beneficial for the delivery of various chemicals and biological agents. These particles have the ability to encapsulate a drug, or medical or agricultural agent and offer controlled and site-specific delivery, as well as co-delivery and sequential release of cosmetic, therapeutic, diagnostic, agricultural, and nutraceutical drugs or agents. Examples of agents may be cosmetic, therapeutic, diagnostic, agricultural, and nutraceutical agents, including proteins, small molecules, vitamins, drugs, imaging agents, as well as perfluorocarbons, oxygen, gases, or drugs, such as active pharmaceutical ingredients (APIs).

Nano- and microparticles have been manufactured using cyclodextrins. Cyclodextrins (CDs) are cyclic molecules formed by (1,4)-linked α-D(+)-glucopyranoside units (FIG. 1A). The most common types are α-, β-, and γ-CDs, comprising 6, 7 and 8 α-D(+)-glucopyranoside units, respectively. CD molecules are shaped like truncated cones with the primary hydroxyl groups at the narrow edges and secondary hydroxyl groups at the wider edges. The inner cavities of CDs are more hydrophobic and outer peripheries are more hydrophilic. Chemically modified CDs have been synthesized by substituting the hydroxyl groups with various other functional groups. CDs can encapsulate hydrophobic molecules into their inner cavities.

By functionalizing the outer shell or inner core of the CDs, the functionality of the CDs can be adapted, designed and optimized for encapsulation and release of a particular drug or agent, or a particular use. However, it is difficult to manufacture particles of functionalized CDs that are stable, non-toxic, biocompatible and biodegradable.

Oxygen, a highly desirable molecule for therapeutic and cosmetic applications, provides a variety of beneficial functions for skin and other tissues. Oxygen can affect adenosine triphosphate (ATP) production via oxidative phosphorylation in the mitochondria, providing energy for cellular functions and protein synthesis. Primarily, the level of physiological oxygen can affect the production of various extracellular matrix molecules by the skin fibroblast cells. Oxygen can stimulate the biosynthesis of collagen, hyaluronic acid, and proteoglycans from normal skin and wounds. Furthermore, oxygen can promote keratinocyte differentiation and migration, which are essential in stratum corneum formation. In wound healing, oxygen is involved in the re-epithelialization, oxidative killing of bacteria, angiogenesis and collagen synthesis. The amount and penetration depth of oxygen diffusion within human dermal tissue are dependent on oxygen pressure gradient and the solubility of oxygen in the tissue.

The supply of oxygen to the skin is not only influenced by internal transport as a result of cutaneous circulation, but also by external transcutaneous diffusion of atmospheric oxygen. Greater oxygen partial pressure on the skin surface can induce higher oxygen flux and penetration depth into the skin. Nevertheless, the stratum corneum presents a permeability barrier to oxygen diffusion from the atmosphere into the deep skin layers, which are composed of keratin proteins, interstitial lipids and partially dehydrated epidermal cells in a closed-packed array structure. Therefore, a topical oxygen treatment that allows a high skin absorption and sustained release of highly concentrated oxygen is a promising approach to increase the oxygen availability and improve skin health.

Perfluorocarbons (PFCs) are organic molecules in which all of the carbon-bound hydrogen atoms are substituted with fluorine. Due to the chemical and thermal stability of the carbon-fluorine bonds, PFC-based materials are particularly attractive in therapeutical applications. One of the unique properties of liquid PFCs is that respiratory gases, especially oxygen, can be dissolved in the “molecular cavities” of the PFCs without engaging in a chemical reaction. For instance, at standard temperature and pressure, the solubility of oxygen in perfluoro-octyl bromide (PFOB) and perfluorodecalin (PFD) is 44.0 mmol/L and 35.5 mmol/L, respectively. On the other hand, under the same conditions water can only dissolve 2.2 mmol/L of oxygen. The oxygen diffusivity in PFCs is also increased compared to water or cell culture media.

PFC-oxygen formulations can be beneficial in a variety of medical applications where oxygen delivery is desired. For example, PFCs have been used to deliver oxygen to hypoxic environment for prolonged cell survival and cancer treatments. However, due to their nonpolar molecular structure, PFCs are very poorly soluble in water or aqueous solutions. Low water solubility of PFCs makes it challenging to administer them alone in aqueous formulations. Therefore, specially designed formulations and delivery devices are necessary for successful introduction of PFCs into biological systems.

Administration of an agent such as a vitamin, protein, sensing molecule, diagnostic agent, drug or active pharmaceutical ingredient via the skin may be transdermal or intradermal (also referred to as local or dermal). Transdermal administration involves transport through the skin such that a therapeutic or diagnostic amount of the agent is achieved in the systemic blood circulation. Intradermal or topical administration of an agent such as a vitamin, protein, sensing or diagnostic agent, drug or active pharmaceutical ingredient involves entry of the agent across the stratum corneum for a cutaneous or local skin effect; that is the pharmacological effect of the agent is localized to the intracutaneous regions of drug penetration and deposition. Preferably, intradermal absorption occurs with little or no systemic absorption or accumulation. Intradermal absorption of the agent involves partitioning of the agent from the applied vehicle into the stratum corneum; diffusion of the agent through the stratum corneum; and partitioning of the agent from the stratum corneum into the epidermis. In contrast, transdermal absorption further involves diffusion of the agent through the epidermis, and capillary uptake of the agent for circulation in the blood.

Whereas transdermal compositions are intended to deliver an agent such as a vitamin, protein, sensing molecule, diagnostic agent, drug or active pharmaceutical ingredient for systemic circulation, a different composition would be needed to deliver the same agent intracutaneously. Topical formulations that achieve delivery of an agent such as a vitamin, protein, sensing molecule, diagnostic agent, drug or active pharmaceutical ingredient across the stratum corneum and retention of the majority of the agent dermally such that it does not enter the bloodstream in significant amounts are complicated to design and require innovative approaches. Several factors determine the permeability of the skin or of specific layers, in particular the stratum corneum, of the skin to agents such as vitamins, proteins, sensing molecules, diagnostic agents, drugs or active pharmaceutical ingredient. These factors include the characteristics of the skin, the characteristics of the agent (e.g., its size (molecular weight or molecular volume), its lipophilicity/hydrophilicity, its polarity), the dosage of the agent applied, interactions between the agent and the delivery vehicle, interactions between the agent and the skin, and interactions of the agent and the skin in the presence of the ingredients in the delivery vehicle. Penetration enhancers are commonly used in transdermal delivery to achieve penetration of an agent across the stratum corneum typically to provide for systemic delivery of the agent, rather than its retention in the epidermis or dermis. Penetration enhancers may however cause adverse effects on the skin especially after long term treatment of the skin with a penetration enhancer.

A modular delivery system comprising nano- an/or microparticles should also be able to reduce side effects, protect labile agents from degradation, and provide better treatment outcomes. A selection of the materials for the synthesis of a modular nano- or microparticle is crucial in order to engineer robust modular systems with inherent beneficial characteristics. At the same time, modular systems should be capable of engaging in physicochemical interactions with the encapsulated agents.

### SUMMARY OF THE INVENTION

It is an object of the present invention to at least partly overcome the above-mentioned problems, and to provide an improved compound and delivery system for administration of one or more agent and/or drug to a subject, such as a human, animal or plant.

In some aspects, the present disclosure is directed to the fabrication and application of a modular delivery system that is capable of encapsulating multiple cosmetic, therapeutic, diagnostic, agricultural, and nutraceutical agents, including proteins, small molecules, vitamins, drugs, imaging agents, as well as other compounds.

This object is achieved by a compound as defined in claim 1.

The compound is a cyclic polysaccharide or a cyclic oligosaccharide compound having structural formula (I):

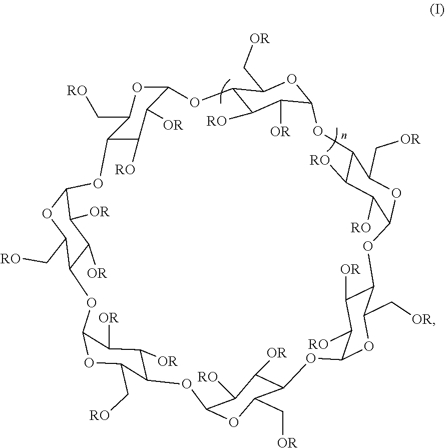

wherein

n is 0, 1, or 2; and,

R is, independently for each occurrence, selected from the group comprising or consisting of H or a radical of polyester, polyethylene glycol, poly(anhydride), polyamide, polyorthoester, poly(L-lactide), poly(D-lactide), poly(D,L-lactide), polyethyleneimine, an oligomer, a protein, a peptide, an antibody, a cell receptor targeting ligand, a fatty acid, a lipid, phenol, a cinnamic acid, a quaternary ammonium group, an amino acid, or co-polymer thereof,

provided at least one instance of R is not H.

The R radical can be varied to obtain compounds having different hydrophobicity for encapsulating different agents as well as for obtaining a compound having different release profiles for the encapsulated agent(s).

In some aspects, the present disclosure is directed to the fabrication and application of a modular delivery system that is capable of encapsulating multiple cosmetic, therapeutic, diagnostic, agricultural, and nutraceutical agents, including proteins, small molecules, vitamins, drugs, imaging agents, as well as other compounds. The compound of the invention is believed to offer controlled and site-specific delivery, as well as co-delivery and sequential release of cosmetic, therapeutic, diagnostic, agricultural, and nutraceutical agents/drugs/molecules. A modular system comprising the compound may reduce side effects, protect labile agents from degradation, and provide better treatment outcomes, and may thus improve patient compliance.

In some aspects, the compound of formula I is biocompatible and biodegradable.

The compounds are believed to be useful for achieving delivery of an agent such as a vitamin, protein, sensing molecule, diagnostic agent, drug or active pharmaceutical ingredient across the stratum corneum and retention of the majority of the agent dermally.

In some aspects, the present invention relates to a cyclic oligosaccharide or a cyclic polysaccharide, wherein the cyclic oligosaccharide or the cyclic polysaccharide is covalently functionalized with at least one radical selected from the group comprising or consisting of radicals of polyester, polyethylene glycol, poly(anhydride), polyamide, polyorthoester, and co-polymer thereof, via the oxygen atom of at least one primary hydroxyl group or at least one secondary hydroxyl group.

In some aspects, the cyclic polysaccharide is a α-, β-, or γ-cyclodextrin comprising 6, 7 and 8 α-D(+)-glucopyranoside units, or a mixture thereof. In some aspects, the cyclic polysaccharide is a 3-cyclodextrin. In some aspects, the cyclic polysaccharide is a α-cyclodextrin.

In some aspects, R is a radical of structural formula (II):

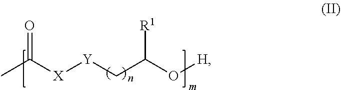

wherein

n is 0 to 3,

m is 2 to 300,000,

X is absent or selected from —CH₂—, —C(H)(OH)—, —O—, —N(H)—, or —C(H)(Hal)-,

Y is absent or selected from —CH₂—, —C(H)(OH)—, —O—, —N(H)—, or —C(H)(Hal)-,

R¹ is H, —OH, alkyl, aryl, or alkenyl, and

Hal is Cl, Br, or I.

The radical of structural formula (II) may have different lengths and substituents. These differences allow for the tuning of the compound of formula I for stable encapsulation of an agent non-covalently bonded to the compound of formula I. The variations in radicals of formula II can also be used to fine tune the release of an agent from the compound or particles made of a plurality of compounds of formula I.

In some aspects, R is a radical of structural formula (II):

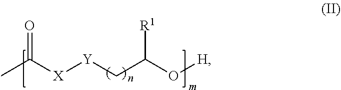

wherein

n is 0 or 2,

m is 50 to 500,

X is absent or —CH₂—,

Y is absent or —CH₂—, and

R¹ is H or alkyl.

In some aspects, R is a polylactone selected from the group comprising caprolactone, valerolactone, glycolide, lactide, ethylglycolide, hexylglycolide and isobutylglycolide, or mixtures thereof. The different functional groups on carpolactones provide for different hydrophobicity for encapsulating different agents giving different release profiles.

In some aspects, the cyclic polysaccharide is a α-, β-, or γ-cyclodextrin comprising 6, 7 and 8 α-D(+)-glucopyranoside units, or a mixture thereof and R is caprolactone or ε-caprolactone. In some aspects, the cyclic polysaccharide is a α-, β-, or γ-cyclodextrin comprising 6, 7 and 8 α-D(+)-glucopyranoside units, or a mixture thereof and R is valerolactone or δ-valerolactone. In some aspects, the cyclic polysaccharide is a α-, β-, or γ-cyclodextrin comprising 6, 7 and 8 α-D(+)-glucopyranoside units, or a mixture thereof and R is lactide. In some aspects, the cyclic polysaccharide is a α- or β-cyclodextrin comprising 6, 7 and 8 α-D(+)-glucopyranoside units, or a mixture thereof and R is caprolactone, valerolactone, glycolide or lactide.

In some aspects, the cyclic polysaccharide is a β-cyclodextrin and the R radical is polycaprolactone (PCL) or poly(ε-caprolactone). PCL is a biodegradable and biocompatible polyester When the R radical is PCL spherical particles can be formed able to encapsulate hydrophobic molecules. PCL polymer covalently bound to CD provides increased stability to the compositions or particles defined herein. PCL polymer has higher stability in physiological conditions compared to other biodegradable polymers (i.e. PLGA and polyurethane). In certain aspects, the weight loss of a PCL-containing particle, for example, a PCL-CD particle, is less than 10% in one-year ex vivo, depending on the physical and biological environment.

The invention also relates to a particle comprising a plurality of cyclic polysaccharide compounds according as defined above, and one or more agent, wherein the plurality of said cyclic polysaccharide compounds form a hollow sphere, and the one or more agent is encapsulated within the hollow sphere; and wherein the one or more agent is non-covalently associated with the cyclic polysaccharide compound. In some aspects, the particle further comprises a surfactant.

Through a selection of the materials for the synthesis of a modular nano- or microparticle a robust modular delivery system can be engineered with inherent beneficial characteristics. The modular delivery systems are believed to be capable of engaging in physicochemical interactions with the encapsulated agents.

In some aspects, the particle defined herein encapsulates one or more agents into two different modules or compartments (one module is the polymeric core, and the other module is the cyclodextrin shell). The design of the shell is also modular, as different types of CDs can be used depending on the intended use. Since different types of CDs with different cavity sizes exist (e.g., α-CD, β-CD, and γ-CD), the selection of the CD type can be customized depending on the size of the molecules that will be encapsulated.

The particles may be used for delivery of an agent such as a vitamin, protein, sensing molecule, diagnostic agent, drug or active pharmaceutical ingredient across the stratum corneum and retention of the majority of the agent dermally. The particles may be used for delivery of oxygen in PCL-containing particles.

In some aspects, the present disclosure relates to a composition, comprising a compound having structural formula (I) or a cyclic oligosaccharide or cyclic polysaccharide and one or more agent, wherein the one or more agent is non-covalently associated with the compound having structural formula (I) or the cyclic oligosaccharide or cyclic polysaccharide.

In certain aspects, the one or more agent is non-covalently associated with the polyester, polyethylene glycol, poly(anhydride), polyamide, polyorthoester, and co-polymer thereof. In some aspects, the one or more agent is non-covalently associated with the cyclic oligosaccharide or the cyclic polysaccharide. In certain aspects, a first agent is non-covalently associated with the polyester, polyethylene glycol, poly(anhydride), polyamide, polyorthoester, and co-polymer thereof; and a second agent is non-covalently associated with the cyclic oligosaccharide or the cyclic polysaccharide.

In some aspects, the particle described herein encapsulates and releases a wide variety of different molecules/agents for cosmetic, medical, diagnostic, agricultural, and nutraceutical applications, such as perfluorocarbons, oxygen, gases, vitamins, proteins, drugs, and imaging agents.

In some aspects, the size of the particles can be finely tuned by varying the formulation and the particle synthesis conditions (including the initial polymer concentration, surfactant concentration, injection rates, and purification methods).

In some aspects, the one or more agent is selected from a group comprising
perfluorocarbon (PFC) selected from the group comprising perfluorooctyl bromide (PFOB), perfluoro(tert-butylcyclohexane), perfluorodecalin (PFD), perfluoroisopropyldecalin, perfluoro-tripropylamine, perfluorotributylamine, perfluoro-methylcyclohexylpiperidine, perfluoro-octylbromide, perfluoro-decylbromide, perfluoro-dichlorooctane, perfluorohexane, dodecafluoropentane, and perfluoro crown ether,
a vitamin selected from the group comprising retinol (vitamin A), vitamin A-propionate, thiamine (vitamin B1), riboflavin (vitamin B2), niacin (vitamin B3), pantothenic acid (vitamin B5), pyridoxine (vitamin B6), biotin (vitamin B7), folic acid (vitamin B9), ascorbic acid (vitamin C), ergocalciferol (vitamin D1), and tocopherols (vitamin E),
a protein selected from the group comprising an enzyme, an antibody, a CAS protein, a transmembrane protein, an amino acid, a cell signaling proteins, and a structural protein such as collagen, hyaluronan, elastin, and tropoelastin,
a fatty acid selected from the group comprising essential, saturated, non-saturated, short chain, medium chain, long chain, very long chain fatty acids, selected but not limited to caprylic acid, capric acid, lauric acid, myristic acid, palmitic acid, stearic acid, arachidic acid, behenic acid, lignoceric acid, cerotic acid, palmitoleic acid, oleic acid, myristoleic acid, linoleic acid, sapienic acid, elaidic acid, vaccenic acid, linoelaidic acid, α-linolenic acid, erucic acid, docosahexaenoic acid, eicosapentaenoic acid, and arachidonic acid.
an imaging agent selected from the group comprising diagnostic imaging agent, a sensing molecule, a contrast agent, a fluorescence sensor, an electrochemical sensor, an electronic sensor, a peptide, an aptamer, a quantum dot, a metallic particle, and a radioisotope of a drug,
genetic material,
insecticide and a pesticide, and
a drug, or any combination thereof.

The drug may be an API, such as the one or more additional drugs listed herein.

In some aspects, the one or more agent is selected from a group comprising vitamins or vitamin A, vitamin A-propionate, vitamin E, rapamycin, oleic acid and BSA protein, perfluorocarbon (PFC) or perfluorooctyl bromide (PFOB) or perfluoro crown ether.

In some aspects, the modular particle system can be used to encapsulate various vitamins. For example, vitamin A is an excellent antioxidant that is widely used for skincare. However, use of vitamin A has limitations due to its susceptibility to degradation when exposed to light or heat. Encapsulation of vitamin A in a particle defined herein can increase its shelf life and delivery efficacy. In some aspects, the particle comprises or consists of the cyclic polysaccharide, which is a α or β-cyclodextrin and the R radical is polycaprolactone (PCL) or polyvalerolactone or polylactide and the agent vitamin A, or vitamin A-propionate or vitamin E. In some aspects, the particle comprises or consists of the cyclic polysaccharide, which is a β-cyclodextrin and the R radical is polycaprolactone (PCL) or polyvalerolactone or polylactide and the agent is vitamin A, or vitamin A-propionate or vitamin E. In some aspects, the particle comprises or consists of the cyclic polysaccharide, which is a β-cyclodextrin and the R radical is polycaprolactone (PCL) and the agent is vitamin A, or vitamin A-propionate or vitamin E. In some aspects, the particle comprises or consists of the cyclic polysaccharide, which is a β-cyclodextrin and the R radical is polycaprolactone (PCL) or poly(ε-caprolactone) and the agent is vitamin A.

In some aspects, the particle comprises or consists of the cyclic polysaccharide, which is a β-cyclodextrin and the R radical is polycaprolactone (PCL) or poly(ε-caprolactone) and the agent is vitamin A-propionate. In some aspects, the particle comprises or consists of the cyclic polysaccharide, which is a β-cyclodextrin and the R radical is polycaprolactone (PCL) or poly(ε-caprolactone) and the agent is vitamin E.

In some aspects, the one or more agent is selected from proteins, such as collagen, elastin, tropoelastin. These proteins are beneficial to the skin and are popular in skin care products. In some aspects, the particle comprises or consists of the cyclic polysaccharide, which is a β-cyclodextrin and the R radical is polycaprolactone (PCL) or poly(ε-caprolactone) and the agent is proteins, such as collagen, elastin, tropoelastin. The stability of the proteins is enhanced by using the particles of the invention for the delivery of the proteins. In some aspects, the particle comprises or consists of the cyclic polysaccharide, which is a α or β-cyclodextrin and the R radical is polycaprolactone (PCL) or polyvalerolactone or polylactide and the agent is rapamycin, oleic acid or BSA protein. In some aspects, the particle comprises or consists of the cyclic polysaccharide, which is a β-cyclodextrin and the R radical is polycaprolactone (PCL) and the agent is rapamycin, oleic acid and BSA protein.

In some aspects, the one or more agent is an imaging agent. Localization of an imaging agent in a specific tissue is desired in medical imaging, and site-specificity can be achieved through the localized delivery of particles encapsulating this type of agents. In some aspects, the particle comprises or consists of the cyclic polysaccharide, which is a β-cyclodextrin and the R radical is polycaprolactone (PCL) or poly(ε-caprolactone) and the agent is an imaging agent.

In some aspects, the one or more agent are perfluorocarbons. Perfluorocarbons (PFC) are a class of agents that can be used for oxygen delivery to the skin and other organs. In some aspects, the (PFC) is PFOB or perfluoro-15-crown-5-ether. In some aspects, the particle optionally further comprises a gas selected from the group comprising oxygen, air, carbon dioxide, and carbon monoxide.

In some aspects, the present disclosure relates to compositions or particles comprising polycaprolactone (PCL) based polymer chemically conjugated with cyclodextrin (CD) for the delivery of oxygenated PFCs, gases, drugs, vitamins, proteins, sensing molecules and diagnostic agents. These compositions or particles described herein can act as local reservoirs of oxygen, PFCs, gases, drugs, vitamins, proteins, sensing molecules and diagnostic agents, and other ingredients.

In certain aspects, the particles as defined herein, loaded with perfluorocarbon, can be used as carriers of oxygen, PFCs, gases, drugs, vitamins, proteins, sensing molecules and diagnostic agents, and other ingredients.

In some aspects, the particle comprises or consists of the cyclic polysaccharide, which is a β-cyclodextrin and the R radical is polycaprolactone (PCL) or poly(ε-caprolactone) and the agent is PFOB, optionally further comprises a gas selected from the group comprising oxygen, air, carbon dioxide, and carbon monoxide. In some aspects, the particle comprises or consists of the cyclic polysaccharide, which is a β-cyclodextrin and the R radical is polycaprolactone (PCL) or poly(ε-caprolactone) and the agent is perfluoro-15-crown-5-ether, optionally further comprises a gas selected from the group comprising oxygen, air, carbon dioxide, and carbon monoxide.

In some aspects, the surfactant is a non-ionic surfactant. In some aspects, the non-ionic surfactant is selected from the group comprising or consisting of D-α-Tocopherol polyethylene glycol 1000 succinate (VETPGS), poly(vinyl acetate) (PVA), TWEEN® 20, TWEEN® 40, TWEEN® 80, POLYSORBATE® 20, POE (4) hydrogenated castor oil, and BRIJ® 96.

In some aspects, the cyclic polysaccharide is a α-, β-, or γ-cyclodextrin comprising 6, 7 and 8 α-D(+)-glucopyranoside units, or a mixture thereof and R is caprolactone or/and the surfactant is VETPGS. In some aspects, the cyclic polysaccharide is a α-, β-, or γ-cyclodextrin comprising 6, 7 and 8 α-D(+)-glucopyranoside units, or a mixture thereof and R is valerolactone or/and the surfactant is VETPGS. In some aspects, the cyclic polysaccharide is a α-, β-, or γ-cyclodextrin comprising 6, 7 and 8 α-D(+)-glucopyranoside units, or a mixture thereof and R is lactide or/and the surfactant is VETPGS.

In some aspects, the cyclic polysaccharide is a α- or β-cyclodextrin comprising 6, 7 and 8 α-D(+)-glucopyranoside units, or a mixture thereof and R is caprolactone, valerolactone or lactides and the surfactant is VETPGS.

The addition of surfactants may be particular useful for delivery of an agent such as a vitamin, protein, sensing molecule, diagnostic agent, drug or active pharmaceutical ingredient and oxygen across the stratum corneum and retention of the majority of the agent dermally.

In some aspects, the surfactant is an anionic surfactant. In some aspects, the anionic surfactant is selected from the group comprising or consisting of sodium cholate, a sulfated natural oil, or cocamidopropyl betaine.

By employing non-ionic or ionic surfactants in the formulation process, the compositions or particles described herein can display a higher capacity to accumulate on the skin and overcome the barrier effects of the stratum corneum, since these surfactant materials can disrupt lipid structures or interact with the intracellular proteins of the stratum corneum.

In some aspects, the particle comprises or consists of the cyclic polysaccharide, which is a α or β-cyclodextrin and the R radical is polycaprolactone (PCL) or polyvalerolactone or polylactide, the surfactant is VETPGS and the agent is PFOB. In some aspects, the particle comprises or consists of the cyclic polysaccharide, which is a β-cyclodextrin, the R radical is polycaprolactone (PCL) or poly(ε-caprolactone), the surfactant is VETPGS and the agent is PFOB. In some aspects, the particle comprises or consists of the cyclic polysaccharide, which is a α or β-cyclodextrin and the R radical is polycaprolactone (PCL) or polyvalerolactone or polylactide, the surfactant is VETPGS and the agent is perfluoro-15-crown-5-ether. In some aspects, the particle comprises or consists of the cyclic polysaccharide, which is a β-cyclodextrin, the R radical is polycaprolactone (PCL) or poly(ε-caprolactone), the surfactant is VETPGS and the agent is perfluoro-15-crown-5-ether. In some aspects, the particle comprises or consists of the cyclic polysaccharide, which is a α or β-cyclodextrin and the R radical is polycaprolactone (PCL) or polyvalerolactone or polylactide, the surfactant is VETPGS and the agent is vitamin A, or vitamin A-propionate or vitamin E. In some aspects, the particle comprises or consists of the cyclic polysaccharide, which is a β-cyclodextrin and the R radical is polycaprolactone (PCL) or polyvalerolactone or polylactide, the surfactant is VETPGS and the agent is vitamin A, or vitamin A-propionate or vitamin E. In some aspects, the particle comprises or consist of the cyclic polysaccharide, which is a β-cyclodextrin, the R radical is polycaprolactone (PCL) or poly(ε-caprolactone), the surfactant is VETPGS and the agent is vitamin A.

In some aspects, the particle comprises further one or more addition drug, and wherein the one or more additional drug is selected from a group comprising or consisting of
antibiotic drug selected from a group comprising penicillins such as penicillin, penicillin G, hetacillin potassium, cloxacillin benzathine, ampicillin and amoxicillin trihydrate, aminocoumarins such as novobiocin, cephalosporins such as cephalexin, ceftiofur sodium, ceftiofur hydrochloride, ceftiofur crystalline free acid, macrolides such as tildipirosin, tylosin, tulathromycin, erythromycin, clarithromycin, and azithromycin, quinolones and fluoroquinolones such as enrofloxacin, ciprofloxacin, levofloxacin, and ofloxacin, sulfonamides such as sulfadimethoxine, co-trimoxazole and trimethoprim, tetracyclines such as tetracycline, oxytetracycline and doxycycline, aminoglycosides such as dihydrostreptomycin sulfate, neomycin, gentamicin and tobramycin, lincosamides such as pirlimycin hydrochloride, lincomycin, clindamycin, and pirlimycin, and amphenicols such as florfenicol,
an antiparasitic drug selected from a group comprising antiprotozoals such as melarsoprol, eflornithine, metronidazole, tinidazole, miltefosine, antihelminthics such as mebendazole, pyrantel pamoate, thiabendazole, diethylcarbamazine, ivermectin, aticestodes such as niclosamide, praziquantel, albendazole, antitrematodes such as praziquantel, antiamoebics such as rifampin and amphotericin B, and broad-spectrum drugs such as nitazoxanide,
an antimycotic drug selected from a group comprising polyenes such as amphotericin b, candicidin, filipin, hamycin, natamycin, nystatin, and rimocidin; azoles such as imidazole, triazole, thiazole, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, and abafungin; allylamines such as amorolfin, butenafine, naftifine, and terbinafine; echinocandins such as anidulafungin, caspofungin and micafungin; and others such as aurones, benzoic acid, ciclopirox olamine, flucytosine or 5-fluorocytosine, griseofulvin, haloprogin, tolnaftate, undecylenic acid, triacetin, crystal violet, castellani's paint, orotomide (f901318), miltefosine, potassium iodide, coal tar, copper(ii) sulfate, selenium disulfide, sodium thiosulfate, piroctone olamine, iodoquinol, clioquinol, acrisorcin, zinc pyrithione, and sulfur,
a coloring agent or dye selected from a group comprising Quinoline yellow, Ponceau 4R, Carmoisine, Patent Blue V, Greens S, Brilliant Blue FCF, Indigotine, Fast Green FCF, Erythrosine, Sunset Yellow, Allura Red AC, Tartrazine, Sunset Yellow FCF, Spirulina, and Betanin,
analgesic or anti-inflammatory drug selected from a group comprising aspirin, ibuprofen, and naproxen, naproxen sodium, diclofenac, acetoaminophen, celecoxib, piroxicam, indomethacin, meloxicam, ketiprofen, sulindac, diflunisal, nabumetone, oxaprozin, tolmetin, salsalate, etodolac, fenoprofen, flurbiprofen, ketorolac, meclofenamate, and mefenamic acid,
a corticosteroid selected from a group comprising prednisone, betamethasone, cortisone, dexamethasone, hydrocortisone, methylprednisolone, prednisolone and triamcinolone acetonide.
an anti-acid drug selected from a group comprising nizatidine, famotidine, cimetidine, ranitidine, omeprazole, esomeprazole, lansoprazole and sodium bicarbonate,
a diuretic selected from a group comprising chlorthalidone, chlorothiazide, hydrochlorothiazide, indapamide, metolazone, amiloride hydrochloride, spironolactone, triamterene, furosemide, and bumetanide,
beta blocker drug selected from a group comprising acebutolol, atenolol, betaxolol, bisoprolol fumarate, carteolol hydrochloride, metoprolol tartrate, metoprolol succinate, nadolol, penbutolol sulfate, pindolol, propranolol hydrochloride, solotol hydrochloride, and timolol maleate,
a ACE inhibitor drug selected from a group comprising benazepril hydrochloride, captopril, enalapril maleate, fosinopril sodium, lisinopril, moexipril, perindopril, quinapril hydrochloride, ramipril, trandolapril,
angiotensin II receptor blocker selected from a group comprising candesartan, eprosartan mesylate, irbesartan, losartan potassium, telmisartan and valsartan
a calcium channel blocker selected from a group comprising amlodipine besylate, bepridil, diltiazem hydrochloride, felodipine, isradipine, nicardipine, nifedipine, nisoldipine, verapamil and hydrochloride,
an alpha blocker selected from a group comprising doxazosin mesylate, prazosin hydrochloride and terazosin hydrochloride,
an alpha-2 receptor agonist, such as methyldopa,
statin selected from a group comprising atorvastatin, fluvastatin, lovastatin, pravastatin, simvastatin and pitavastatin,
a PCSK9 inhibitor selected from a group comprising evolocumab and alirocumab,
chemotherapic drugs selected from a group comprising 5-fluorouracil, 6-mercaptopurine, cytarabine, gemcitabine, and methotrexate, paclitaxel and rapamycin,
an immunotherapeutic drug selected from a group comprising ipilimumab, nivolumab, pembrolizumab, atezolizumab, avelumab and durvalumab,
genetic material selected from a group comprising single stranded DNA, double stranded DNA, plasmid DNA, siRNA, shRNA, gRNA, sgRNA, tRNA and mRNA,
a pesticide selected from a group comprising herbicide, insecticides, nematicide, molluscicide, piscicide, avicide, rodenticide, bactericide, insect repellent, animal repellent, antimicrobial, and fungicide.

In some aspects, the particles as defined herein can encapsulate different cargos in the cyclodextrin and polymer regions, which allows for dual-release from the outer shell followed by the release from the core. The different cargoes can potentially be released in different environments and at different time points. These cargos can include oxygen, PFCs, gases, drugs, vitamins, proteins, sensing molecules, diagnostic agents and other ingredients.

In some aspects, compositions or particles described herein comprise a polymeric core that encapsulates the first active agent, and cyclodextrin (CD) outer shell that can be loaded with the second active agent. In particular, the polymeric core comprises polycaprolactone (PCL), which is covalently conjugated with CD.

In some aspects, the delivery system that contains two active agents in different moieties of the compositions or particles described herein can be capable of releasing both agents in a time- and sequence-dependent manner. On the treatment site, the second active agent loaded in the CD outer shell is released during the early stage, followed by the release of the first active agent from the polymeric core. The release of two agents in combination can offer synergistic efficacy to the target site.

In some aspects, the particle comprises further an addition drug, and wherein the one or more additional drug is selected from a group comprising or consisting of antibiotic drugs selected from a group comprising or consisting of penicillins, aminocoumarins, cephalosporins, macrolides, quinolones and fluoroquinolones, sulfonamides, tetracyclines and amphenicols,

antiparasitic drug selected from a group comprising or consisting of antiprotozoals, antihelminthics, aticestodes, antiamoebics and nitazoxanide,

antimycotic drug selected from a group comprising or consisting of amphotericin b, candicidin, azoles allylamines and echinocandins,

analgesic or anti-inflammatory drug selected from a group comprising or consisting of aspirin, ibuprofen, and naproxen, diclofenac, acetoaminophen, celecoxib, piroxicam, indomethacin, meloxicam, ketiprofen, sulindac, diflunisal, nabumetone, oxaprozin, tolmetin, salsalate, etodolac, fenoprofen, flurbiprofen, ketorolac, meclofenamate, and mefenamic acid,

corticosteroid selected from a group comprising or consisting of prednisone, betamethasone, cortisone, dexamethasone, hydrocortisone, methylprednisolone, prednisolone and triamcinolone acetonide,

anti-acid drug selected from a group comprising or consisting of nizatidine, famotidine, cimetidine, ranitidine and omeprazole,

diuretic selected from a group comprising or consisting of chlorthalidone, chlorothiazide and bumetanide,

beta blocker drug selected from a group comprising or consisting of acebutolol, atenolol, betaxolol, bisoprolol fumarate, carteolol hydrochloride, metoprolol tartrate, metoprolol succinate, nadolol, penbutolol sulfate, pindolol, propranolol hydrochloride, solotol hydrochloride and timolol maleate,

ACE inhibitor drug selected from a group comprising or consisting of benazepril hydrochloride, captopril and enalapril,

angiotensin II receptor blocker selected from a group comprising or consisting of candesartan, eprosartan mesylate and irbesartan,

calcium channel blocker selected from a group comprising or consisting of amlodipine besylate and bepridil,

alpha blocker selected from a group comprising or consisting of doxazocin mesylate, prazosin hydrochloride and statin,

PCSK9 inhibitor selected from a group comprising or consisting of evolocumab and alirocumab,

chemotherapic drugs selected from a group comprising or consisting of 5-fluorouracil, 6-mercaptopurine, cytarabine, gemcitabine, and methotrexate, paclitaxel and rapamycin,

immunotherapeutic drug selected from a group comprising or consisting of ipilimumab, nivolumab, pembrolizumab, atezolizumab, avelumab and durvalumab,

genetic selected from a group comprising or consisting of single stranded DNA, double stranded DNA, plasmid DNA, siRNA, shRNA, gRNA, sgRNA, tRNA and mRNA,

pesticide selected from a group comprising or consisting of herbicide, insecticides, bactericide, insect repellent, animal repellent, antimicrobial, and fungicide.

The invention also relates to a method of making the cyclic polysaccharide compound as defined herein, comprising:

a) providing a first solution comprising said cyclic polysaccharide compound and a catalyst;

b) adding a monomer of the radical R to the first solution, thereby providing a reaction mixture;

c) stirring or mixing the reaction mixture at a temperature for a period of time; and

d) isolating the cyclic oligosaccharide or cyclic polysaccharide compound.

In some aspects, the present disclosure relates to a one-pot catalytic approach for the preparation of novel polycaprolactone (PCL) based polymer chemically conjugated with cyclodextrin (CD) (PCL-CD).

In some aspects, the present disclosure relates to direct catalytic modification for the generation of the PCL-CD, which is further engineered into compositions or particles described herein for the encapsulation of various agents.

The invention also relates to a method of making the particles as defined herein, comprising

e) providing a second solution comprising the cyclic polysaccharide compound as defined herein or as made using the method defined above, the one or more agent, and a second solvent;

f) providing a third solution comprising a third solvent and optionally a surfactant;

g) contacting the second solution with the third solution, wherein
the second solution contacts the third solution in a microfluidic reactor,
the second solution contacts the third solution under ultrasonic treatment, or
the second solution contacts the third solution by mechanical stirring; and

h) removing the second solvent and the third solvent by evaporation or dialysis.

The polymeric system may comprise a cyclodextrin (hydrophilic) region on the outer shell and a biodegradable PCL polymer (hydrophobic) region in the core. This drug delivery system can encapsulate different cargos in the cyclodextrin and polymer regions, which also allows for dual-release and/or sequential release from the outer shell followed by the release from the core. The particles defined herein comprising the biodegradable PCL-CD will undergo chemical bond degradations in slightly acidic conditions (pH ^{˜}5.5) and in the presence of digestive enzymes on the skin. For example, PCL is susceptible to hydrolysis by the proteolytic enzymes at the dermal-epidermal junction. In one aspect, the particles defined herein are used to encapsulate perfluorocarbons that can dissolve high level of oxygen, such that oxygen can be delivered in different biological environments. The tunable hydrophobicity and size of the particles can display high affinity to the lipid composition on stratum corneum, resulting in consistent and controlled release of oxygen, and this can eventually provide higher and prolonged oxygen partial pressure to the skin cells.

In some aspects, the particles defined herein comprise a core-shell structure with a hollow filled with PFC (PCL-CD/PFC). These structures result in stabilizing the water-insoluble cargoes, such as PFCs, and promote better dispersion of the cargo in aqueous solutions and topical creams or gels.

The hydrophobic cargos can be encapsulated in the CD moiety of the composition or particle of the present disclosure by resuspending the cargos and PCL-CD in an organic solvent followed by removal of the solvent. For example, water-insoluble resveratrol can be mixed with PCL-CD suspension in DMSO at room temperature. Since the CD portion can entrap hydrophobic ingredients, resveratrol can be encapsulated once the DMSO is removed by dialysis.

The invention further relates to a cosmetic, pharmaceutical, or nutraceutical composition comprising the particles as defined herein, together with a pharmaceutically, cosmetically, or nutraceutically acceptable carrier.

In some aspects, the cosmetic, pharmaceutical, agricultural and nutraceutical composition is adapted for administration to a subject orally, by inhalation spray, parenterally, topically, rectally, nasally, buccally, vaginally or via an implanted reservoir. In some aspects, the cosmetic, pharmaceutical, agricultural and nutraceutical composition is adapted for administration topically, transdermally or interdermally to a subject.

The invention also relates to a cosmetic, pharmaceutical, or nutraceutical composition defined herein for use as dietary supplement, food additive, agricultural applications to plants or to ground/soil, as a blood substitute. The invention also relates to a cosmetic, pharmaceutical, or nutraceutical composition defined herein for use in prevention and/or treatment of a disease in a mammal.

In some aspects, the pharmaceutical composition is for use of delivering oxygen, carbon monoxide, carbon dioxide or air to a subject. In some aspects, the pharmaceutical composition is for use of delivering oxygen.

In some aspects, the pharmaceutical composition is for use as a blood substitute. The composition or particles as defined herein can be used for transport and delivery of oxygen via blood to organs and the like in a body of a mammal. The composition or particles can thus be used to assist or collaborate with the red blood cells in the blood of a mammal.

In some aspects, the present disclosure relates to a method of supplementing the oxygen-carrying capacity of a subject's blood, comprising administering to the subject an effective amount of the blood substitute.

In some aspects, the cosmetic, pharmaceutical, or nutraceutical composition comprising the particles as defined herein, and a pharmaceutically, cosmetically, or nutraceutically acceptable carrier, comprise or consist of the particles comprising or consisting of the cyclic polysaccharide, which is a β-cyclodextrin and the R radical is polycaprolactone (PCL) or poly(ε-caprolactone) and the agent is PFOB or perfluoro-15-crown-5-ether, and further comprises a gas selected from the group comprising oxygen, air, carbon dioxide, and carbon monoxide, for use of delivering oxygen, carbon monoxide, carbon dioxide or air to a subject, as a blood substitute.

The invention further relates to a cosmetic, pharmaceutical, or nutraceutical composition defined above for use in prevention and/or treatment of a skin disease in a mammal.

The invention also relates to a cosmetic, pharmaceutical, or nutraceutical composition defined herein, wherein the one or more agent and/or drug is a pesticide selected from a group comprising or consisting of herbicide, insecticides, nematicide, molluscicide, piscicide, avicide, rodenticide, bactericide, insect repellent, animal repellent, antimicrobial, and fungicide, for use in treating plants. Such a composition may be administered to or on the plant or to, in or on the soil in the proximity (ca 0.001 to 2 meter distance) of the plant.

### BRIEF DESCRIPTION OF THE DRAWINGS

The invention will now be explained more closely by the description of different embodiments of the invention and with reference to the appended figures.

FIG. 1A is a scheme showing the cone-structure as a simplified representative of the CD structures.

FIG. 1B is a scheme showing the one-pot catalytic reaction for the preparation of the covalently conjugated PCL-β-CD by mixing the CD and the ε-caprolactone in the presence of an organocatalyst (1,5,7-Triazabicyclo[4.4.0]dec-5-ene) in DMSO at 80° C. for 24 h.

FIG. 1C is a scheme showing the possibilities of preparing CD covalently functionalized with various moieties such as polymers, oligomers or small molecules through known chemistries such as esterification.

FIG. 2A is a scheme showing a simplified representative view of the cavity of the CD structures.

FIG. 2B is a scheme showing a simplified representative view of the cavity of CD functionalized with a polymer, such as PCL. In a polar environment, such as an aqueous solution, a hydrophobic polymer attached to the CD is localized in the hydrophobic cavity of the CD molecule.

FIG. 2C is a scheme CD covalently functionalized with various moieties such as polymers, oligomers or small molecules. In a polar environment, such as an aqueous solution, a hydrophobic moiety is localized in the hydrophobic cavity of the CD molecule.

FIG. 3 is a schematic representation of a particle comprising a plurality of functionalized CD compounds and an encapsulated agent, such as a PCL-CD particle with the hydrophilic cup-shaped cyclodextrin forming the outer layer of the shell and the hydrophobic PCL tails forming the inner layer of the shell. The core is the encapsulated hydrophobic agent, such as PFOB.

FIG. 4 shows a typical proton nuclear magnetic resonance (¹H-NMR) spectrum of the PCL-β-CD.

FIG. 5 is the Differential Scanning calorimetry (DSC) plot of the PCL-β-CD showing a glass transition temperature of 42.2° C. and a melting point around 57° C.

FIG. 6 is a graph showing the cartridge with microfluidic channels employed in a Nanoassemblr® reactor.

FIG. 7 shows a typical ¹⁹F-NMR spectrum of PCL-β-CD/PFOB dissolved in ethyl acetate-d₈ with 2,2,2-trifluoroacetic acid as an internal reference.

FIG. 8A is a bar graph showing the influence of polymer concentration on the hydrodynamic size and polydispersity index (PDI) of PFOB-loaded PCL-β-CD particles (formulated with the following parameters; Nanoassemblr®: 1 or 3% PCL-β-CD and 3 or 9% PFOB in ethyl acetate as the organic phase, 1% Vitamin E d-α-Tocopheryl polyethylene glycol 1000 succinate (VETPGS) surfactant in water as the aqueous phase, 9:1 aqueous to organic flow rate ratio, and 20 mL/min total flow rate).

FIG. 8B is a bar graph showing the influence of total flow rate ratio on the hydrodynamic size and PDI of PFOB-loaded PCL-β-CD particles (formulated with the following parameters; Nanoassemblr®: 3% PCL-β-CD and 9% PFOB in ethyl acetate as the organic phase, 1% VETPGS surfactant in water as the aqueous phase, 9:1 aqueous to organic flow rate ratio, and 12 or 20 mL/min total flow rate).

FIG. 8C is a bar graph showing the influence of total flow rate ratio on the hydrodynamic size and PDI of PFOB-loaded PCL-β-CD particles (formulated with the following parameters; Nanoassemblr®: 3% PCL-β-CD and 3 or 9% PFOB in ethyl acetate as the organic phase, 1% VETPGS surfactant in water as the aqueous phase, 9:1 aqueous to organic flow rate ratio, and 20 mL/min total flow rate).

FIG. 9A is the transmission electron microscope (TEM) images showing the morphology of PFOB-loaded PCL-β-CD particles (formulated with the following parameters; Nanoassemblr®: 3% PCL-β-CD and 9% PFOB in ethyl acetate as the organic phase, 1% VETPGS surfactant in water as the aqueous phase, 9:1 aqueous to organic flow rate ratio, and 20 mL/min total flow rate). The image showing the surface of the particle.

FIG. 9B the TEM image showing an internal hollow core surrounded by a polymer shell.

FIG. 10 is an image depicting the system for ultrasonicated formulation of PCL-β-CD/PFOB particles. The microtip was placed in the center of 10 mL solution of surfactant, and the polymer solution was added dropwise under sonication.

FIG. 11 is a microscope images showing the morphology of PFOB-loaded PCL-β-CD particles (formulated with the following parameters by solvent: 3% PCL-β-CD with 15% PFOB in ethyl acetate as the organic phase, 1% VETPGS surfactant in water as the aqueous phase).

FIG. 12 is a bar graph showing the cell viability of human epidermal keratinocytes cells after treated with PFOB-loaded PCL-β-CD particles for 48 h (formulated with the following parameters; Nanoassemblr®: 3% PCL-β-CD and 9% PFOB in ethyl acetate as the organic phase, 1% VETPGS surfactant in water as the aqueous phase, 9:1 aqueous to organic flow rate ratio, and 20 mL/min total flow rate).

FIG. 13 is a bar graph showing the dissolved oxygen level of water, 1.5% PFD, and 0.3% PCL-R-CD particles loaded with 1.5% PFOB (formulated with the following parameters by solvent evaporation method; 3% PCL-β-CD and 15% PFOB in ethyl acetate as the organic phase, 1% VETPGS surfactant in water as the aqueous phase, 9:1 aqueous to organic volume ratio) over 5 h at 30° C. The 1.5 PFD group is shown as competitor group.

FIG. 14A is a microscope images showing the morphology of the PCL-CD/PFOB microparticles (formulated as presented in Example 8: conditions: 400 rpm, 40° C., 5 min stirring). Demonstrating microparticles of 10-25 μm sizes.

FIG. 14B is a microscope images showing the morphology of the PCL-CD/PFOB microparticles (formulated as presented in Example 8: conditions: 400 rpm, 40° C., 5 min stirring). Demonstrating microparticles of 10-25 μm sizes.

FIG. 14C is a microscope images showing the morphology of the PCL-CD/PFOB microparticles (formulated as presented in Example 8: conditions: 400 rpm, 40° C., 21 min stirring). Demonstrating microparticles of 1-10 μm sizes.

FIG. 15A is the scanning electron microscope (SEM) images showing the morphology of the PCL-CD/PFOB microparticles (formulated as presented in Example 8: conditions: 400 rpm, 40° C., 21 min stirring). Scale bar 40 μm.

FIG. 15B is the scanning electron microscope (SEM) images showing the morphology of the PCL-CD/PFOB microparticles (formulated as presented in Example 8: conditions: 400 rpm, 40° C., 21 min stirring). Scale bar 20 μm.

FIG. 15C is the scanning electron microscope (SEM) images showing the morphology of the PCL-CD/PFOB microparticles (formulated as presented in Example 8: conditions: 400 rpm, 40° C., 21 min stirring). Scale bar 20 μm.

FIG. 15D is the scanning electron microscope (SEM) images showing the morphology of the PCL-CD/PFOB microparticles (formulated as presented in Example 8: conditions: 400 rpm, 40° C., 21 min stirring). Scale bar 50 μm.

FIG. 16 is dynamic light scattering (DLS) images showing the sizes and size distribution of the PCL-CD/PFOB microparticles (formulated as presented in Example 8: conditions: 400 rpm, 40° C., 21 min stirring).

FIG. 17 is a microscope images showing the morphology of the PCL-CD/Vitamin A-propionate microparticles (formulated as presented in Example 9: conditions: 400 rpm, 40° C., 21 min stirring). Demonstrating microparticles of 1 μm sizes.

FIG. 18 is the scanning electron microscope (SEM) images showing the morphology of the PCL-CD/Vitamin A-propionate microparticles (formulated as presented in Example 9: conditions: 400 rpm, 40° C., 21 min stirring). Scale bar 50 μm.

FIG. 19 is dynamic light scattering (DLS) images showing the sizes and size distribution of the PCL-CD/Vitamin A-propionate microparticles (formulated as presented in Example 9: conditions: 400 rpm, 40° C., 21 min stirring). The sizes of the particles observed ranged from greater than 1 μm to less than 10 μm.

FIG. 20 is dynamic light scattering (DLS) images showing the sizes and size distribution of the PCL-CD/PFOB Nanoparticles (formulated as presented in Example 10). The sizes of the particles observed ranged from greater than 400 nm to less than 1 μm.

FIG. 21 is dynamic light scattering (DLS) images showing the sizes and size distribution of the PCL-CD/Vitamin A-propionate Nanoparticles (formulated as presented in Example 11. The sizes of the particles observed ranged from greater than 300 nm to less than 500 nm.

FIG. 22 is the ¹H-NMR spectrum of the retinyl propionate nanoparticles within the modular nanoparticle delivery system presenting the encapsulation efficiency. DMF was used as an internal standard (reference) and DMSO-d₆ as the solvent for the ¹H-NMR experiment demonstrating encapsulation efficiency of 42.7% (formulated as presented in Example 11).

FIG. 23 is absorbance spectra demonstrating the determination of loading efficacy of protein (Bovine Serum Albumin) using within the nanoparticle delivery system using Nanodrop spectrophotometer (Presented in Example 12).

FIG. 24 shows a typical proton nuclear magnetic resonance (¹H-NMR) spectrum of the α-cyclodextrin-L-lactide.

FIG. 25 shows a typical proton nuclear magnetic resonance (¹H-NMR) spectrum of the α-cyclodextrin-δ-valerolactone.

FIG. 26 shows a typical proton nuclear magnetic resonance (¹H-NMR) spectrum of the β-cyclodextrin-δ-valerolactone.

FIG. 27 shows a typical proton nuclear magnetic resonance (¹H-NMR) spectrum of the Oleic acid nanoparticles encapsulated within the nanoparticle delivery system as in Example 13.

FIG. 28 is absorbance spectra demonstrating the determination of the release of the protein (Bovine Serum Albumin) using within the nanoparticle delivery system using Nanodrop spectrophotometer (Presented in Example 13).

FIG. 29 shows a typical proton nuclear magnetic resonance (¹H-NMR) spectrum of the β-cyclodextrin-L-lactide.

### DETAILED DESCRIPTION OF VARIOUS EMBODIMENTS OF THE INVENTION

### Definitions

The definitions set forth in this application are intended to clarify terms used throughout this application. The term “herein” means the entire application.

As used herein, the term “perfluorocarbon” (PFC) refers to an organic molecule in which all carbon-bound hydrogen atoms are replaced with fluorine atoms.

As used herein, “polymer” or “polymer radical” refers to a chemical species or a radical made up of repeatedly linked moieties. In some embodiments, the number of repeatedly linked moieties range between approximately 2-300,000. The linked moieties may be identical in structure or may have variation of moiety structure. In some embodiments, the polymer is made up of moieties linked by ester groups, referred to as “polyester”. “Polylactone” refers to a polyester from a cyclic ester, such as caprolactone, valerolactone, glycolide (the diester of glycolic acid), lactide (the diester of 2-hydroxypropionic acid), ethylglycolide, hexylglycolide, and isobutylglycolide.

As used herein, “poly mide” refers to polymers comprising amide bonds/peptide bonds that is formed when an amine group of one amino acid forms a bond with the carboxylic group of another amino acid, resulting in the loss of a water molecule.

As used herein, “polyanhydrate/polyanhydride” refers to biodegradable polymers created from repeating monomers connected by anhydride bonds (a functional group characterized by two acyl groups merged by an oxygen atom with the formula (RC(O))₂O).

As used herein, “oligomer” refers to a molecule with few repeating units of the corresponding monomer (normally having a repeating unit between about five and hundreds) in difference with polymers comprising a large number of repeating units.

As used herein, “lipid” refers to molecules that are made up mostly of hydrocarbons and are hydrophobic, nonpolar and not soluble in water but soluble in nonpolar solvents, examples of lipids include fats, oils, waxes, hormones and certain vitamins.

As used herein, “phenol” refers to a family of organic compounds characterized by a hydroxyl (—OH) group attached to a carbon atom that is part of an aromatic ring.

As used herein, “fatty acid” refers to a carboxylic acid with a long aliphatic chain that is either saturated or unsaturated.

As used herein, “an amino acid” refers to a compound comprising a basic amino group (—NH₂), an acidic carboxyl group (—COOH), and an organic R group (or side chain) that is unique to each amino acid.

As used herein, “a quaternary ammonium group” refers to positively charged ions with the structure NR₄⁺ with N being a nitrogen atom and R being alkyl or aryl group consisting of carbon and hydrogen atoms arranged in a chain. They are permanently charged, independently of the acidity of their solution.

As used herein, “an antibody” refers to a class of protein called an immunoglobulin that are made of specialized white blood cells to identify and neutralize material foreign to an immune system.

As used herein, “co-polymer thereof” refers to a polymer formed when two (or more) types of monomers are linked in the same polymer chain, or the link of two or more polymers together.

The term “alkyl” as used herein is a branched or unbranched saturated hydrocarbon group of 1 to 24, or 1 to 10, or 1 to 4 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched (i.e., linear). The alkyl group can also be substituted or unsubstituted (preferably unsubstituted). For example, the alkyl group can be substituted with one or more groups including, but not limited to, alkyl, cycloalkyl, alkoxy, amino, ether, halide, hydroxy, nitro, silyl, sulfoxo, sulfonate, carboxylate, or thiol, as described herein. A “lower alkyl” group is an alkyl group containing from one to six (e.g., from one to four) carbon atoms.

The term “alkenyl”, as used herein, refers to an aliphatic group containing at least one carbon-carbon double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, an alkenyl group may be substituted by one or more alkyl, carbocyclyl, aryl, heterocyclyl, or heteroaryl groups is contemplated. Exemplary alkenyl groups include, but are not limited to, vinyl (—CH═CH₂), allyl (—CH₂CH═CH₂), cyclopentenyl (—C₅H₇), and 5-hexenyl (—CH₂CH₂CH₂CH₂CH═CH₂).

The term “aryl,” alone or in combination, as used herein, means a carbocyclic aromatic system containing one or more rings, which may be attached together in a pendent manner or may be fused. In particular embodiments, aryl is one, two or three rings. In one aspect, the aryl has five to twelve ring atoms. The term “aryl” encompasses aromatic radicals such as phenyl, naphthyl, tetrahydronaphthyl, indanyl, biphenyl, phenanthryl, anthryl and acenaphthyl. An “aryl” group can have 1 to 4 substituents, such as alkyl, hydroxyl, halo, haloalkyl, nitro, cyano, alkoxy, alkylamino and the like.

As used herein, the term “derivative” refers to molecules in which at least one hydrogen atom of is replaced with a substituent. In the case of an oxo substituent (“═O”), two hydrogen atoms are replaced. When substituted, one or more of the groups below are “substituents.”

Substituents include, but are not limited to, halogen, hydroxy, oxo, cyano, nitro, amino, alkylamino, dialkylamino alkyl, alkoxy, alkylthio, haloalkyl, aryl, arylalkyl, heteroaryl, heteroarylalkyl, heterocycle, and heterocyclealkyl, as well as, —NRₐR_{b}, —NRₐC(═O)R_{b}, —NRₐC(═O)NRₐNR_{b}, —NRₐC(═O)R_{b}—NRₐSO₂R_{b}, —C(═O)Rₐ, C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. In addition, the above substituents may be further substituted with one or more of the above substituents, such that the substituent comprises a substituted alkyl, substituted aryl, substituted arylalkyl, substituted heterocycle, or substituted heterocyclealkyl. Rₐ and R_{b} in this context may be the same or different and, independently, hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl.

As used herein, the term “drug” refers to any agent capable of having a physiologic effect (e.g., a therapeutic or prophylactic effect) on a biosystem such as prokaryotic or eukaryotic cells or organisms, in vivo or in vitro. The drug can be selected from a variety of known classes of drugs, including, for example, analgesics, anesthetics, anti-inflammatory agents, anthelmintics, anti-arrhythmic agents, antiasthma agents, antibiotics (including penicillins), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, proteins, antibodies, and xanthines.

As used herein, the term “protein” or “peptide” refers to any large biomolecules and macromolecules that contain at least one polypeptide chains. The protein of interest can be selected from various proteins with different functions, including enzymes, antibodies, transmembrane proteins, cell signaling proteins (including insulin), and structural proteins (including collagen).

As used herein, the term “diagnostic” or “sensing molecules” refers to an organic or inorganic compound that is used for imaging, diagnostic, and sensing purposes. The “diagnostic” or “sensing molecules” can be selected from a variety of diagnostic agents, including diagnostic imaging agents, sensing molecules, contrast agents, fluorescence sensors, electrochemical sensors, electronic sensors, peptides, aptamers, quantum dots, metallic particles, radioisotopes (such as fluorine-18), as well as other molecules.

Examples of suitable isotopes that may be incorporated include ²H (also written as “D” for deuterium), ³H (also written as “T” for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is used will depend on the specific application of that radio-labelled derivative. For example, for in vitro receptor labelling and competition assays, compounds that incorporate ³H or ¹⁴C are often useful. For radio-imaging applications ¹¹C or ¹⁸F are often useful.

As used herein, term “cosmetic ingredient” refers to any organic or inorganic ingredients used for cosmetic purposes. Cosmetic ingredient can be selected from a variety of vitamins, proteins, perfluorocarbons, minerals, oils, emulsifiers, emollients, surfactants, lipids, polymers as well as other compounds used for cosmetic applications.

The term “vitamin” as used herein refers to an organic compound required by an organism as a vital nutrient in limited amounts. An organic chemical compound (or related set of compounds) is called a vitamin when it cannot be synthesized in sufficient quantities by an organism and must be obtained from the diet. Thus, the term is conditional both on the circumstances and on the particular organism. For example, ascorbic acid (vitamin C) is a vitamin for humans, but not for most other animals, and biotin (vitamin H) and vitamin D are required in the human diet only in certain circumstances. By convention, the term vitamin includes neither other essential nutrients, such as dietary minerals, essential fatty acids, or essential amino acids (which are needed in larger amounts than vitamins), nor the large number of other nutrients that promote health but are otherwise required less often. Thirteen vitamins are universally recognized for humans at present: Vitamins A, C, D, E, K, the B vitamins (thiamine, riboflavin, niacin, pantothenic acid, biotin, vitamin B6, vitamin B12, and folate), as well as others.

As used herein, the term “surfactant” refers to its usual meaning. A long list of relevant surfactants and related surfactant definitions is provided in EP0475160 and U.S. Pat. No. 6,165,500 which are incorporated herein by reference in their entirety. Therefore, the following list only offers a selection, which in no way is complete or exclusive, of various kinds of surfactants that are particularly common or useful in conjunction with the present patent application. This includes long chain fatty alcohols or ionized long chain fatty acids, long chain fatty ammonium salts, such as alkyl- or alkenoyl-trimethyl-, -dimethyl- and -methyl-ammonium salts, alkyl- or alkenoyl sulfate salts, long fatty chain dimethyl aminoxides, such as alkyl- or alkenoyl dimethyl aminoxides, long fatty chains, such as, for example, alkanoyl, dimethyl aminoxides and especially long fat dodecyl dimethyl aminoxide, long fatty chains, as per for example, alkyl-N-methylglucamides and alkanoyl-N-methylglucamides, such as MEGA-8®, MEGA-9® and MEGA-10®, N-long fat chain-N, N-dimethylglycins, for example N-alkyl-N, N-dimethylglycins, 3-(long-chain fatty acid dimethylammonium) alkanesulfonates, for example, 3-(acyldimethylammonium) alkanesulfonates, long-chain fatty acid derivatives of sulphosuccinate salts, such as bis (2-ethyl alkyl) sulfosuccinate salts, chain sulfobetaines long fat, for example acyl sulfobetaines, betaines of long chain, such as EMPIGEN BB® or ZWITTERGENT-3-16®, -3-14, -3-12, -3-10, or -3-8, or polyethylene glycol acylphenyl ethers, especially nonaethylene glycol octylphenyl ether, long-chain polyethylene fatty ethers, especially polyethylene acyl ethers, such as nonaethylene decyl ether, nonaethylene dodecyl ether or octaethylene dodecyl ether, polyethylene glycol isoacyl ether, such as octaethylene glycol isotridecyl ether, chain esters polyethylene glycol sorbitan grease, for example polyethylene glycol sorbitan-acyl esters and especially polyethylene glycol monolaurate (for example TWEEN 20®), polyethylene glycol sorbitan monooleate (for example TWEEN 80°), polyethylene glycol sorbitan monolaurolelate, polyethylene glycero monostolate, sorbitan polyethylene glycol sorbitan monoelaidate, polyethylene glycol sorbitan miristolelate, polyethylene glycol sorbitan palmitoleinylate, polyethylene glycol sorbitan petrosellinilate, polyhydroxyethylene long chain fatty ethers, by example polyhydroxyethylene acyl ethers, such as polyhydroxyethylene lauryl ethers, polyhydroxyethylene myristoyl ethers, polyhydroxyethylene cetyl stearyl, polyhydroxyethylene palmityl ethers, polyhydroxyethylene oleoyl ethers, polyhydroxyethylene ethylene oxoethylene, polyhydroxyethylene, polyhydroxyethylene, polyhydroxyethylene, polyhydroxyethylene, polyhydroxyethylene, polyhydroxyethylene, polyhydroxyethylene ether, or 10-, or 12-lauryl, myristoyl, palmitoyl, palmitoleil, oleoyl or linoeyl ethers (Brij® series), or in the corresponding esters, polyhydroxyethylene laureate, -myristate, -palmitate, -stearate or -oleate, especially polyhydroxyethylene-8-stearate (Myrj 45®) and polyhydroxyethylene-8-oleate, castor oil polyethoxylate 40 (Cremophor EL®), mono sorbitan long chain fat, for example alkylate (Arlacel® or Span® series), such as sorbitan-monolaureate (Arlacer 20®, Span 20®) or monooleate, long fatty chains, for example acyl-N-methylglucamides, alkanoyl-N-methylglucamides, especially decanoyl-N-methylglucamide, dodecanoyl-N-methylglucamide or octadecanoyl-N-methylglucamide, long-chain fatty sulfates, for example alkyl sulfates, alkyl sulfate salts, such as lauryl sulfate (SDS), oleoyl sulfate; long-chain fatty thioglycosides, such as alkylthioglucosides, for example, heptyl-, octyl-, nonyl- and decyl-beta-D-thioglucopyranoside; long-chain fatty acid derivatives of various carbohydrates, such as pentoses, hexoses and disaccharides, for example, alkyl glucosides and maltosides, such as hexyl-, heptyl-, octyl-, nonyl- and decyl-beta-D-glucopyranoside or D-maltopyranoside; additionally a salt, especially a sodium salt, of cholate, deoxycholate, glycocholate, glycodeoxycholate, taurodeoxycholate, taurocholate, a fatty acid salt, especially oleate, elaidate, linoleate, laurate, or myristate, lysophospholipids, n-octadecyl-glycerophosphatidic acid, octadecyl-phosphoryl glycerol, octadecyl-phosphorylserine or phosphatidylcholine, n-long-chain fatty-glycerol-phosphatidic acids, such as n-acyl-glycero-phosphatidic acids, especially glycidic acid, especially oleoyl-glycero-phosphatidic, n-long chain fat-phosphorylglycerol, such as n-acyl-phosphorylglycerol, especially lauryl-, miristoyl-, oleoyl- or palmitoleoyl-phosphorylglycerol, n-long chain fat-phosphorylserine, such as n-acyl phosphorylserine, especially lauryl-, myristoyl, oleoyl or palmitoleoyl-phosphorylserine, n-tetradecyl-glycero acid-phosphatidic, n-tetradecyl-fosforilglicerol, n-tetradecyl-fosfori Serine, the corresponding elaidoyl-, vaccenyl-lysophospholipids, the corresponding short chain phospholipids, as well as all surfactant polypeptides. Surfactant chains are typically selected to be in a fluid state or at least to be compatible with maintaining the fluid state of the chain in carrier aggregates.

As used herein, the term “natural oil” refers to an oil derived from a plant, yeast, or animal source. The term “natural oil” includes natural oil derivatives, unless otherwise indicated. The sources can be modified plant, yeast, or animal sources (e.g., genetically modified plant, yeast, or animal sources), unless indicated otherwise. Examples of natural oils include, but are not limited to, vegetable oils, algae oils, fish oils, animal fats, tall oils, derivatives of these oils, combinations of any of these oils, and the like. Representative non-limiting examples of vegetable oils include canola oil, rapeseed oil, coconut oil, corn oil, cottonseed oil, olive oil, palm oil, peanut oil, safflower oil, sesame oil, soybean oil, sunflower oil, linseed oil, palm kernel oil, tung oil, jatropha oil, mustard oil, penny cress oil, camelina oil, and castor oil. Representative non-limiting examples of animal fats include lard, tallow, poultry fat, yellow grease, and fish oil. Tall oils are by-products of wood pulp manufacture.

As used herein, the term “nanoparticle” refers to a particle having a characteristic dimension of less than about 1 micrometer and at least about 1 nanometer, where the characteristic dimension of the particle is the smallest cross-sectional dimension of the particle (such as diameter, length, or width).

As used herein, the term “microparticle” refers to a particle having a characteristic dimension of less than about 1 millimeter and at least about 1 micrometer, where the characteristic dimension of the particle is the smallest cross-sectional dimension of the particle (such as diameter, length, or width).

The average particle size as used herein is defined as being the particle size (z average) measured using dynamic light scattering (DLS), which is also known as photon correlation spectroscopy (PSC) or quasi-elastic light scattering (QELS). The particle size measured thereby is also frequently called hydrodynamic diameter and reflects how a particle diffuses within a fluid. The measured hydrodynamic diameter is equivalent to that of an ideal sphere having the same translational diffusion coefficient as the particle being measured. Since the surface structure may have a significant influence, the hydrodynamic diameter measured using DLS can be significantly larger than the true diameter measured e.g. by electron microscopy.

In Dynamic Light Scattering (DLS), the polydispersity index (PDI) reflects the width of the particle size distribution. It ranges from 0 to 1. A value of zero refers to an ideal suspension with no distribution in size. Distributions with PDI values of 0.1 or smaller are called monodisperse while dispersions with values between 0.1 and 0.3 are considered as having a narrow size distribution. Dispersions having a PDI larger than 0.5 are considered being polydisperse.

As used herein, the term “biodegradable”, refers to the ability of a compound, particle, or material, to undergo degradation in a biological system, for example enzymatic degradation or chemical degradation.

The terms “carrier” or “pharmaceutically acceptable carrier”, or “cosmetically acceptable carrier” or “nutraceutically acceptable carrier”, as used herein, refer to a medium that is used to prepare a desired dosage form of a compound or particle. A pharmaceutically, cosmetically, and/or nutraceutically acceptable carrier can include one or more solvents, diluents, or other liquid vehicles; dispersion or suspension aids; surface active agents; isotonic agents; thickening or emulsifying agents; preservatives; solid binders; lubricants; and the like. Remington's Pharmaceutical Sciences, Fifteenth Edition, E. W. Martin (Mack Publishing Co., Easton, Pa., 1975) and Handbook of Pharmaceutical Excipients, Third Edition, A. H. Kibbe ed. (American Pharmaceutical Assoc. 2000), disclose various carriers used in formulating pharmaceutical compositions and known techniques for the preparation thereof. Pharmaceutically acceptable carriers that may be used in the disclosed compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

“Topical administration” is used in its conventional sense to mean delivery of a topical vitamin, protein, sensing molecule, diagnostic agent, drug or pharmacologically active agent to the skin or mucosa, as in, for example, the treatment of various skin disorders. Topical administration, in contrast to transdermal administration, provides a local rather than a systemic effect as agents do not cross stratum corneum.

“Transdermal” delivery of an agent such as a vitamin, protein, sensing molecule, diagnostic agent, drug or pharmacologically active agent is meant administration of the agent to the skin surface of an individual so that the agent passes through the skin tissue and into the individual's blood stream, thereby providing a systemic effect. The term “transdermal” is intended to include “transmucosal” drug administration, i.e., administration of the agent to the mucosal (e.g., sublingual, buccal, vaginal, rectal) surface of an individual so that the agent passes through the mucosal tissue and into the individual's blood stream.

The present invention relates to a cyclic polysaccharide compound having structural formula (I):

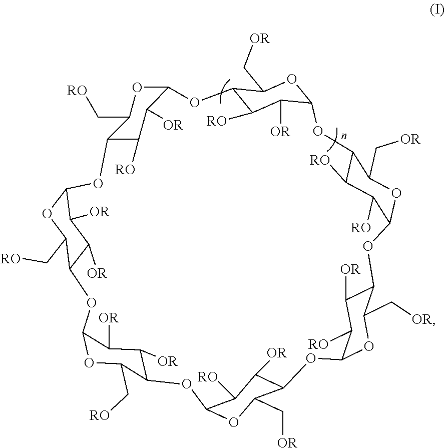

wherein

n is 0, 1, or 2; and,

R is, independently for each occurrence, H or a radical of polyester, polyethylene glycol, poly(anhydride), polyamide, polyorthoester, poly(L-lactide), poly(D-lactide), poly(D,L-lactide), polyethyleneimine, and co-polymer thereof, an oligomer, a protein, a peptide, an antibody, a cell receptor targeting ligand, a fatty acid, a lipid, phenol, a cinnamic acid, a quaternary ammonium group, an amino acid, or co-polymer thereof, provided at least one instance of R is not H.

The cyclic polysaccharide may be a α-, β-, or γ-cyclodextrin comprising 6, 7 and 8 α-D(+)-glucopyranoside units, or a mixture thereof. The cyclic polysaccharide may be a β-cyclodextrin. The cyclic polysaccharide may be a α-cyclodextrin.

The cyclic polysaccharide compound having structural formula (I) may have an R which are each independently radicals of polyester, polyethylene glycol, poly(anhydride), polyamide, polyorthoester, or co-polymer thereof, provided at least one instance of R is not H.

Preferably, at least two instances of R, are polyester radicals.

The R radical may be a radical of structural formula (II):

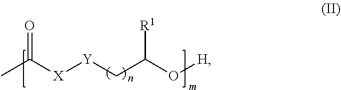

wherein

n is 0 to 3,

m is 2 to 300,000,

X is absent or selected from —CH₂—, —C(H)(OH)—, —O—, —N(H)—, or —C(H)(Hal)-,

Y is absent or selected from —CH₂—, —C(H)(OH)—, —O—, —N(H)—, or —C(H)(Hal)-,

R¹ is H, —OH, alkyl, aryl, or alkenyl, and

Hal is Cl, Br, or I.

Or the R radical may be a radical of structural formula (II), wherein

n is 0, 1 or 2,

m is 2 to 100,000,

X is absent or —CH₂—,

Y is absent or —CH₂—, and

R¹ is H, —OH, alkyl.

Or the R radical may be a radical of structural formula (II), wherein

n is n is 0 or 2,

m is 20 to 1000,

X is absent or —CH₂—,

Y is absent or —CH₂—, and

R¹ is H, —OH or —CH₃.

The R radical may be a polyester, which may be a polylactone. The polylactone may be selected from the group comprising or consisting of valerolactone, glycolide, lactide, ethylglycolide, hexylglycolide and isobutylglycolide, or mixtures thereof. The R radical may be poly(ε-caprolactone), poly(δ-valerolactone), poly(ε-valerolactone), polyglycolide, or polylactide. The R radical may be poly(E caprolactone). The R radical may be poly(ε-valerolactone). The R radical may be lactide.

Preferably, the oxygen atom of at least two hydroxyl groups are functionalized with a polyester radical.

The cyclic oligosaccharide or cyclic polysaccharide may be further functionalized with a quaternary ammonium group.

The molecular weight of each polyester radical may be independently from about 100 to about 300,000 or from about 500 to about 80,000. The molecular weight of the cyclic oligosaccharide or cyclic polysaccharide may be from about 2000 to about 7,200,000.

The cyclic polysaccharide may be a α-, β-, or γ-cyclodextrin comprising 6, 7 and 8 α-D(+)-glucopyranoside units, or a or β-cyclodextrin, wherein n is 0, 1, or 2; and R is a radical of structural formula (II) wherein

n is 0 to 3,

m is 2 to 300,000,

X is absent or selected from —CH₂—, —C(H)(OH)—, —O—, —N(H)—, —C(H)(Hal)-,

Y is absent or selected from —CH₂—, —C(H)(OH)—, —O—, —N(H)—, —C(H)(Hal)-,

R¹ is H, —OH, alkyl, aryl, or alkenyl, and

Hal is Cl, Br, or I, or

the R radical may be a radical of structural formula (II), wherein

n is 0, 1 or 2,

m is 2 to 100,000,

X is absent or —CH₂—,

Y is absent or —CH₂—, and

R¹ is H, —OH, alkyl, or

the R radical may be a radical of structural formula (II), wherein

n is n is 0 or 2,

m is 20 to 1000,

X is absent or —CH₂—,

Y is absent or —CH₂—, and

R¹ is H, —OH or —CH₃.

The cyclic polysaccharide may be a α-, β-, or γ-cyclodextrin comprising 6, 7 and 8 α-D(+)-glucopyranoside units, or α, or β-cyclodextrin, wherein n is 0, 1, or 2; and wherein R is a polylactone selected from the group comprising or consisting of valerolactone, glycolide, lactide, ethylglycolide, hexylglycolide and isobutylglycolide, or mixtures thereof, or

R is poly(ε-caprolactone), poly(δ-valerolactone), poly(ε-valerolactone), polyglycolide, or polylactide.

The cyclic polysaccharide may be a α-, β-, or γ-cyclodextrin comprising 6, 7 and 8 α-D(+)-glucopyranoside units, or a or β-cyclodextrin, wherein n is 0, 1, or 2; and wherein R is poly(ε caprolactone).

The cyclic polysaccharide may be a α-, β-, or γ-cyclodextrin comprising 6, 7 and 8 α-D(+)-glucopyranoside units, or α or β-cyclodextrin, wherein n is 0, 1, or 2; and wherein R is poly(6-valerolactone). The cyclic polysaccharide may be a α-, β-, or γ-cyclodextrin comprising 6, 7 and 8 α-D(+)-glucopyranoside units, or a or 3-cyclodextrin, wherein n is 0, 1, or 2; and wherein R is polylactide.

The invention also relates to a method of making the cyclic polysaccharide compound as defined above, comprising or consisting the steps of

a) providing a first solution comprising said cyclic polysaccharide compound and a catalyst;

b) adding a monomer of the radical R to the first solution, thereby providing a reaction mixture;

c) mixing or stirring the reaction mixture at a temperature for a period of time; and

d) isolating the compound or the cyclic oligosaccharide or cyclic polysaccharide.

wherein the temperature is from about 24° C. to about 300° C.

The temperature in step c) may be from about 24° C. to about 300° C., or from about 5° C. to about 200° C., or from about 50° C. to about 100° C., from room temperature to 90° C., or about 80° C.

The temperature in steps a), b) and d) may be room temperature, i.e. between 15 and 26° C.

The period of time in step c) may be from 15 minutes to 30 hours, or from 10 hours to 24 hours.

The first solution may comprise a first solvent. The first solvent may be selected from the group comprising or consisting of dimethyl sulfoxide (DMSO), dimethylformamide (DMF), N-methyl-2-pyrrolidone (NMP), acetonitrile (MeCN), acetone, ethyl acetate (EtOAc), dichloromethane (DCM), tetrahydrofuran (THF), or a mixture thereof. The first solvent may be DMSO.

The catalyst may be a Brønsted acid, a Lewis acid, a metal catalyst, and organocatalyst, or an enzyme. The catalyst may be selected from the group comprising or consisting of 1,5,7-triazabicyclo[4.4.0]dec-5-ene (TBD), amino acid, oligopeptide, tartaric, acid, lactic acid, citric acid, fumaric acid, malic acid, α-hydroxy-substituted carboxylic acid, sulfonic acid, tetrazole, trifluoromethanesulfonic acid, HCl.Et₂O, diphenyl phosphate, γ-resorcylic acid, methanesulfonic acid, Candida antarctica Lipase B, or Sn(OTf)₂. The catalyst may be TBD.

The invention also relates to a particle comprising or consisting a plurality of cyclic polysaccharide compounds as defined above, wherein the plurality of cyclic polysaccharide compounds form a hollow sphere. Optionally, the particle further comprises a surfactant.

The particles may further comprise or consist of one or more agent or drug, wherein the one or more agent or drug is encapsulated within the hollow sphere; and wherein the agent is non-covalently associated with the cyclic polysaccharide compound. Optionally the particle further comprises a surfactant.

The one or more agent may be encapsulated within the hollow sphere and an additional agent or drug as defined herein, may be present in the outer shell of the cyclic polysaccharide. The agent and an additional agent or drug may be encapsulated within the hollow sphere. The agent may be a drug encapsulated within the hollow sphere. One or more agent may be encapsulated within the hollow sphere with no other agent or drug present in or on the particle.

The amount of the one or more agent in the particle or composition may be from about 10 wt. % to about 90 wt. %, or about 55 wt. %, wherein wt. % are percentages of the total weight of the particles. The diameter of the particle may be from 50 nm to 20,000 nm, or from 500 nm to 2,000 nm or about 800 nm.

The invention relates to a method of making the particles defined above, comprising or consisting of

e) providing a second solution comprising the cyclic polysaccharide compound as defined above, the one or more agent, and a second solvent;

f) providing a third solution comprising a third solvent and optionally a surfactant;

g) contacting the second solution with the third solution, wherein
the second solution contacts the third solution in a microfluidic reactor,
the second solution contacts the third solution under ultrasonic treatment, or
the second solution contacts the third solution by mechanical stirring; and

h) removing the second solvent and the third solvent by evaporation or dialysis.

The second solvent may be selected from the group comprising or consisting of EtOAc, DCM, chloroform, diethyl ether, ethanol, methanol, isopropanol, butanol, toluene, DMF, THF, acetone, MeCN, dioxane, NMP, ethylene glycol, pyridine, propylene glycol, methyl isobutyl ketone, methyl isopropyl ketone, DMSO, or a mixture thereof. The second solvent may be EtOAc.

The third solvent may be selected from the group comprising or consisting of water, ethanol, glycerol, DCM, chloroform, diethyl ether, methanol, isopropanol, butanol, toluene, DMF, THF, acetone, acetonitrile, dioxane, NMP, ethylene glycol, pyridine, propylene glycol, methyl isobutyl ketone, methyl isopropyl ketone, DMSO, phosphate buffered saline (pH 7.4), Tris buffer (pH 8.0), or a mixture thereof. The third solvent may be water.

The w/v concentration of the compound or the cyclic oligosaccharide or cyclic polysaccharide in the second solution may be from about 0.5% to about 10%, or from about 1% to about 3%.

In step f) may be provided a third solution comprising a third solvent and a surfactant. The surfactant may be a non-ionic surfactant or an anionic surfactant. The non-ionic surfactant may be selected from the group comprising or consisting of D-α-Tocopherol polyethylene glycol 1000 succinate (VETPGS), poly(vinyl acetate) (PVA), TWEEN® 20, TWEEN®40, TWEEN® 80, POLYSORBATE®20, POE (4) hydrogenated castor oil, and BRIJ® 96. The non-ionic surfactant may be VETPGS. The ionic surfactant may be selected from the group comprising or consisting of sodium cholate, a sulfated natural oil, or cocamidopropyl betaine. The weight per volume (w/v) concentration of the surfactant in the third solution is from about 0.1% to about 10%, or from about 0.1% to about 5%, or from about 0.5% to about 2%, or about 1%.

The w/v concentration of the one or more agent in the second solution may be from about 1% to about 30%, or from about 3% to about 17%, or from about 9% to about 15%.

The one or more agent may be selected from the group comprising or consisting of a perfluorocarbon (PFC), a drug, a vitamin, a protein, a fatty acid, an imaging agent, genetic material, a pesticide and a combination thereof.

The one or more agent may be a vitamin selected from the group comprising or consisting of retinol (vitamin A), vitamin A-propionate, thiamine (vitamin B1), riboflavin (vitamin B2), niacin (vitamin B3), pantothenic acid (vitamin B5), pyridoxine (vitamin B6), biotin (vitamin B7), folic acid (vitamin B9), ascorbic acid (vitamin C), ergocalciferol (vitamin D1), and tocopherols (vitamin E). The one agent may be vitamin A.

The particle may comprise or consist of cyclic polysaccharide, which may be α-, β-, or γ-cyclodextrin comprising 6, 7 and 8 α-D(+)-glucopyranoside units, or a or β-cyclodextrin, wherein n is 0, 1, or 2; and wherein R is a radical of structural formula (II) wherein

n is 0 to 3,

m is 2 to 300,000,

X is absent or selected from —CH₂—, —C(H)(OH)—, —O—, —N(H)—, —C(H)(Hal)-,

Y is absent or selected from —CH₂—, —C(H)(OH)—, —O—, —N(H)—, —C(H)(Hal)-,

R¹ is H, —OH, alkyl, aryl, or alkenyl, and

Hal is Cl, Br, or I, or

the R radical may be a radical of structural formula (II), wherein

n is 0, 1 or 2,

m is 2 to 100,000,

X is absent or —CH₂—,

Y is absent or —CH₂—, and

R¹ is H, —OH, alkyl, or

the R radical may be a radical of structural formula (II), wherein

n is n is 0 or 2,

m is 20 to 1000,

X is absent or —CH₂—,

Y is absent or —CH₂—, and

R¹ is H, —OH or —CH₃, and the one agent is vitamin A, vitamin A-propionate or vitamin E.

The particle may comprise or consist of cyclic polysaccharide, which may be α-, β-, or γ-cyclodextrin comprising 6, 7 and 8 α-D(+)-glucopyranoside units, or a or β-cyclodextrin, wherein n is 0, 1, or 2; and wherein R is a polylactone selected from the group comprising or consisting of valerolactone, glycolide, lactide, ethylglycolide, hexylglycolide and isobutylglycolide, or mixtures thereof, or

R is poly(ε-caprolactone), poly(δ-valerolactone), poly(ε-valerolactone), polyglycolide, or polylactide and the one agent is vitamin A, vitamin A-propionate or vitamin E.

The particle may comprise or consist of cyclic polysaccharide, which may be a α-, β-, or γ-cyclodextrin comprising 6, 7 and 8 α-D(+)-glucopyranoside units, or α or β-cyclodextrin, and wherein R is poly(E caprolactone), and the one agent is vitamin A, vitamin A-propionate or vitamin E. The particle may further comprise a surfactant such as VETPGS.

The one or more agent may be a protein selected from the group comprising or consisting of an enzyme, an antibody, a CAS protein, a transmembrane protein, an amino acid, a cell signaling proteins, and a structural protein such as collagen, hyaluronan, elastin, or tropoelastin.

The one or more agent may be a fatty acid selected from the group comprising or consisting of essential, saturated, non-saturated, short chain, medium chain, long chain, very long chain fatty acids, selected but not limited to caprylic acid, capric acid, lauric acid, myristic acid, palmitic acid, stearic acid, arachidic acid, behenic acid, lignoceric acid, cerotic acid, palmitoleic acid, oleic acid, myristoleic acid, linoleic acid, sapienic acid, elaidic acid, vaccenic acid, linoelaidic acid, α-linolenic acid, erucic acid, docosahexaenoic acid, eicosapentaenoic acid, and arachidonic acid.

The one or more agent may be an imaging agent selected from the group comprising or consisting of a diagnostic imaging agent, a sensing molecule, a contrast agent, a fluorescence sensor, an electrochemical sensor, an electronic sensor, a peptide, an aptamer, a quantum dot, a metallic particle, and a radioisotope.

The one or more agent may be rapamycin, oleic acid and BSA protein. The particle may comprise or consist of cyclic polysaccharide, which may be α-, β-, or γ-cyclodextrin comprising 6, 7 and 8 α-D(+)-glucopyranoside units, or α or β-cyclodextrin, wherein n is 0, 1, or 2; and wherein R is a radical of structural formula (II) wherein

n is 0 to 3,

m is 2 to 300,000,

X is absent or selected from —CH₂—, —C(H)(OH)—, —O—, —N(H)—, —C(H)(Hal)-,

Y is absent or selected from —CH₂—, —C(H)(OH)—, —O—, —N(H)—, —C(H)(Hal)-,

R¹ is H, —OH, alkyl, aryl, or alkenyl, and

Hal is Cl, Br, or I, or

the R radical may be a radical of structural formula (II), wherein

n is 0, 1 or 2,

m is 2 to 100,000,

X is absent or —CH₂—,

Y is absent or —CH₂—, and

R¹ is H, —OH, alkyl, or

the R radical may be a radical of structural formula (II), wherein

n is n is 0 or 2,

m is 20 to 1000,

X is absent or —CH₂—,

Y is absent or —CH₂—, and

R¹ is H, —OH or —CH₃, and the one agent is rapamycin, oleic acid and BSA protein.

The particle may comprise or consist of cyclic polysaccharide, which may be α-, β-, or γ-cyclodextrin comprising 6, 7 and 8 α-D(+)-glucopyranoside units, or α or β-cyclodextrin, wherein n is 0, 1, or 2; and wherein R is a polylactone selected from the group comprising or consisting of valerolactone, glycolide, lactide, ethylglycolide, hexylglycolide and isobutylglycolide, or mixtures thereof, or

R is poly(ε-caprolactone), poly(δ-valerolactone), poly(ε-valerolactone), polyglycolide, or polylactide and the one agent is rapamycin, oleic acid and BSA protein.

The particle may comprise or consist of cyclic polysaccharide, which may be a α-, β-, or γ-cyclodextrin comprising 6, 7 and 8 α-D(+)-glucopyranoside units, or α or β-cyclodextrin, and wherein R is poly(ε caprolactone), and the one agent is rapamycin, oleic acid and BSA protein. The particle may further comprise a surfactant such as VETPGS.

The one or more agent may be a PFC selected from the group comprising or consisting of perfluorooctyl bromide (PFOB), perfluoro(tert-butylcyclohexane), perfluorodecalin (PFD), perfluoroisopropyldecalin, perfluoro-tripropylamine, perfluorotributylamine, perfluoro-methylcyclohexylpiperidine, perfluoro-octylbromide, perfluoro-decylbromide, perfluoro-dichlorooctane, perfluorohexane, dodecafluoropentane, and perfluoro crown ether.

The one agent may be PFOB. The one agent may be perfluoro-15-crown-5-ether. The boiling point of the PFC may be from about 50° C. to about 200° C.

The particle may comprise or consist of cyclic polysaccharide, which may be α-, β-, or γ-cyclodextrin comprising 6, 7 and 8 α-D(+)-glucopyranoside units, or β-cyclodextrin, wherein n is 0, 1, or 2; and wherein R is a radical of structural formula (II) wherein

n is 0 to 3,

m is 2 to 300,000,

X is absent or selected from —CH₂—, —C(H)(OH)—, —O—, —N(H)—, —C(H)(Hal)-,

Y is absent or selected from —CH₂—, —C(H)(OH)—, —O—, —N(H)—, —C(H)(Hal)-,

R¹ is H, —OH, alkyl, aryl, or alkenyl, and

Hal is Cl, Br, or I, or

the R radical may be a radical of structural formula (II), wherein

n is 0, 1 or 2,

m is 2 to 100,000,

X is absent or —CH₂—,

Y is absent or —CH₂—, and

R¹ is H, —OH, alkyl, or

the R radical may be a radical of structural formula (II), wherein

n is n is 0 or 2,

m is 20 to 1000,

X is absent or —CH₂—,

Y is absent or —CH₂—, and

R¹ is H, —OH or —CH₃, and the one agent is PFOB or perfluoro-15-crown-5-ether.

The particle may comprise or consist of cyclic polysaccharide, which may be α-, β-, or γ-cyclodextrin comprising 6, 7 and 8 α-D(+)-glucopyranoside units, or α or β-cyclodextrin, wherein n is 0, 1, or 2; and wherein R is a polylactone selected from the group comprising or consisting of valerolactone, glycolide, lactide, ethylglycolide, hexylglycolide and isobutylglycolide, or mixtures thereof, or

R is poly(ε-caprolactone), poly(δ-valerolactone), poly(ε-valerolactone), polyglycolide, or polylactide and the one agent is PFOB or perfluoro-15-crown-5-ether.

The particle may comprise or consist of cyclic polysaccharide, which may be a α-, β-, or γ-cyclodextrin comprising 6, 7 and 8 α-D(+)-glucopyranoside units, or α or β-cyclodextrin, and wherein R is poly(E caprolactone), and the one agent is PFOB or perfluoro-15-crown-5-ether. The particle may further comprise a surfactant such as VETPGS.

The particle may optionally further comprise a gas selected from the group comprising oxygen, air, carbon dioxide, and carbon monoxide, especially when the one agent is a PFC selected from PFOB or perfluoro-15-crown-5-ether. The volume of PFC may be from about 1% to about 95% of the total volume of the particle/composition.

The one or more additional drug may be an antibiotic drug selected from the group comprising or consisting of penicillins such as penicillin, penicillin G, hetacillin potassium, cloxacillin benzathine, ampicillin and amoxicillin trihydrate, aminocoumarins such as novobiocin, cephalosporins such as cephalexin, ceftiofur sodium, ceftiofur hydrochloride, ceftiofur crystalline free acid, macrolides such as tildipirosin, tylosin, tulathromycin, erythromycin, clarithromycin, and azithromycin, quinolones and fluoroquinolones such as enrofloxacin, ciprofloxacin, levofloxacin, and ofloxacin, sulfonamides such as sulfadimethoxine, co-trimoxazole and trimethoprim, tetracyclines such as tetracycline, oxytetracycline and doxycycline, aminoglycosides such as dihydrostreptomycin sulfate, neomycin, gentamicin and tobramycin, lincosamides such as pirlimycin hydrochloride, lincomycin, clindamycin, and pirlimycin, and amphenicols such as florfenicol.

The one or more additional drug may be an antiparasitic drug selected from the group comprising or consisting of antiprotozoals such as melarsoprol, eflornithine, metronidazole, tinidazole, miltefosine, antihelminthics such as mebendazole, pyrantel pamoate, thiabendazole, diethylcarbamazine, ivermectin, aticestodes such as niclosamide, praziquantel, albendazole, antitrematodes such as praziquantel, antiamoebics such as rifampin and amphotericin B, and broad-spectrum drugs such as nitazoxanide.

The one or more additional drug may be an antimycotic drug selected from the group comprising or consisting of amphotericin b, candicidin, filipin, hamycin, natamycin, nystatin, and rimocidin; azoles such as imidazole, triazole, thiazole, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, and abafungin; allylamines such as amorolfin, butenafine, naftifine, and terbinafine; echinocandins such as anidulafungin, caspofungin and micafungin; and others such as aurones, benzoic acid, ciclopirox olamine, flucytosine or 5-fluorocytosine, griseofulvin, haloprogin, tolnaftate, undecylenic acid, triacetin, crystal violet, castellani's paint, orotomide (f901318), miltefosine, potassium iodide, coal tar, copper(ii) sulfate, selenium disulfide, sodium thiosulfate, piroctone olamine, iodoquinol, clioquinol, acrisorcin, zinc pyrithione, and sulfur.

The one or more additional drug may be a coloring agent or dye selected from the group comprising or consisting of Quinoline yellow, Ponceau 4R, Carmoisine, Patent Blue V, Greens S, Brilliant Blue FCF, Indigotine, Fast Green FCF, Erythrosine, Sunset Yellow, Allura Red AC, Tartrazine, Sunset Yellow FCF, Spirulina, and Betanin.

The one or more additional drug may be an analgesic or anti-inflammatory drug selected from the group comprising or consisting of aspirin, ibuprofen, and naproxen, naproxen sodium, diclofenac, acetoaminophen, celecoxib, piroxicam, indomethacin, meloxicam, ketiprofen, sulindac, diflunisal, nabumetone, oxaprozin, tolmetin, salsalate, etodolac, fenoprofen, flurbiprofen, ketorolac, meclofenamate, and mefenamic acid.

The one or more additional drug may be a corticosteroid drug selected from the group comprising or consisting of prednisone, betamethasone, cortisone, dexamethasone, hydrocortisone, methylprednisolone, prednisolone and triamcinolone acetonide.

The one or more additional drug may be an anti-acid drug selected from the group comprising or consisting of nizatidine, famotidine, cimetidine, ranitidine, omeprazole, esomeprazole, lansoprazole and sodium bicarbonate.

The one or more additional drug may be diuretic drug selected from the group comprising or consisting of chlorthalidone, chlorothiazide, hydrochlorothiazide, indapamide, metolazone, amiloride hydrochloride, spironolactone, triamterene, furosemide, and bumetanide.

The one or more additional drug may be a beta blocker drug selected from the group comprising or consisting of acebutolol, atenolol, betaxolol, bisoprolol fumarate, carteolol hydrochloride, metoprolol tartrate, metoprolol succinate, nadolol, penbutolol sulfate, pindolol, propranolol hydrochloride, solotol hydrochloride, and timolol maleate.

The one or more additional drug may be an ACE inhibitor drug selected from the group comprising or consisting of benazepril hydrochloride, captopril, enalapril maleate, fosinopril sodium, lisinopril, moexipril, perindopril, quinapril hydrochloride, ramipril, trandolapril.

The one or more additional drug may be an angiotensin II receptor blocker selected from the group comprising or consisting of candesartan, eprosartan mesylate, irbesartan, losartan potassium, telmisartan and valsartan.

The one or more additional drug may be a calcium channel blockers drug selected from the group comprising or consisting of amlodipine besylate, bepridil, diltiazem hydrochloride, felodipine, isradipine, nicardipine, nifedipine, nisoldipine, verapamil and hydrochloride.

The one or more additional drug may be an alpha blocker drug selected from the group comprising or consisting of doxazocin mesylate, prazosin hydrochloride and terazosin hydrochloride.

The one or more additional drug may be an alpha-2 receptor agonist drug, which may be methyldopa.

The one or more additional drug may be a combined alpha and beta-blocker drug selected from the group comprising or consisting of carvedilol and labetalol hydrochloride.

The one or more additional drug may be a central agonists drug selected from the group comprising or consisting of alpha methyldopa, clonidine hydrochloride, guanabenz acetate and guanfacine hydrochloride.

The one or more additional drug may be a peripheral adrenergic inhibitor selected from the group comprising or consisting of guanadrel, guanethidine monosulfate and reserpine.

The one or more additional drug may be selected from hydralazine hydrochloride and minoxidil.

The one or more additional drug may be a statin drug selected from the group comprising or consisting of atorvastatin, fluvastatin, lovastatin, pravastatin, simvastatin and pitavastatin.

The one or more additional drug may be a PCSK9 inhibitor drug selected from the group comprising or consisting of evolocumab and alirocumab.

The one or more additional drug may be a chemotherapy drug selected from the group comprising or consisting of 5-fluorouracil, 6-mercaptopurine, cytarabine, gemcitabine, and methotrexate, paclitaxel, rapamycin, among others.

The one or more additional drug may be an immunotherapeutic drug selected from the group comprising or consisting of ipilimumab, nivolumab, pembrolizumab, atezolizumab, avelumab, durvalumab, among others.

The one or more additional drug may be genetic material selected from the group comprising or consisting of single stranded DNA, doule stranded DNA, plasmid DNA, siRNA, shRNA, gRNA, sgRNA, tRNA, mRNA.

The one or more additional drug may be a pesticide selected from the group comprising or consisting of herbicide, insecticides, nematicide, molluscicide, piscicide, avicide, rodenticide, bactericide, insect repellent, animal repellent, antimicrobial, and fungicide.

The particles may further comprise or consist of a surfactant, which may be a non-ionic surfactant or an anionic surfactant. The non-ionic surfactant may be selected from the group comprising or consisting of D-α-Tocopherol polyethylene glycol 1000 succinate (VETPGS), poly(vinyl acetate) (PVA), TWEEN® 20, TWEEN® 40, TWEEN® 80, POLYSORBATE® 20, POE (4) hydrogenated castor oil, and BRIJ® 96. The non-ionic surfactant may be VETPGS. The ionic surfactant may be selected from the group comprising or consisting of sodium cholate, a sulfated natural oil, or cocamidopropyl betaine. The weight per volume (w/v) concentration of the surfactant in the third solution is from about 0.1% to about 10%, or from about 0.1% to about 5%, or from about 0.5% to about 2%, or about 1%.

### Formulation

The invention also relates to a cosmetic, pharmaceutical, agricultural or nutraceutical composition comprising the particles as defined herein, and a pharmaceutically, cosmetically, or nutraceutically acceptable carrier. For topical administration, a lotion or cream may be used. Examples of acceptable carrier may be diluent, humectant, thickener, solvent, preservative, antioxidant, stabilizer, emulsifier, emollient, flavoring agents, pH adjuster, lubricants, suspending agents, binders, or tablet disintegrating agents. The diluent may be a solvent or water. The humectant may be glycerin, sodium hyaluronate, phenoxyethanol, hydrolyzed hyaluronic acid, propanediol or a mixture hereof. The thickener may be xanthan gum, hydroxyethyl acrylate/sodium acryloyldimethyl taurate copolymer, or a mixture hereof. The preservative may be caprylyl glycol, ethylhexylglycerin, benzyl alcohol, ethylhexylglycerin, tocopherol or a mixture hereof. The antioxidant may be pentaerythrityl tetra-di-t-butyl hydroxyhydrocinnamate, tocopheryl acetate, or mixtures thereof. The stabilizer may be cetearyl alcohol. The emulsifier may be cetearyl olivate, sorbitan olivate, glyceryl stearate, cetearyl alcohol, sorbitan stearate, or mixtures thereof. The emollient may be coco-caprylate/caprate, dicaprylyl carbonate, squalane, or mixtures thereof.

When using more than one agent or drug, the cosmetic, pharmaceutical, agricultural or nutraceutical composition may comprise or consist of (i) the particles as defined herein, (ii) an additional agent or drug and (iii) one or more pharmaceutically acceptable carrier. Alternatively, the composition may comprise or consist of (i) the particles as defined herein, and one or more acceptable carrier (ii) an additional agent or drug and one or more acceptable carrier.

Such conjoint treatment may be achieved by way of the simultaneous, sequential or separate dosing of the individual components to a mammal, plant or soil.

The cosmetic, pharmaceutical, agricultural or nutraceutical composition may be a solid, a semi-solid, a plaster, a solution, a pill, a capsule, a tablet, a food additive, a spray, a suspension, a lotion, a cream, a foam, a gel, a paste, or an emulsion. The cosmetic, pharmaceutical or nutraceutical composition may be adapted for administering the composition orally, by inhalation spray, parenterally, topically, rectally, nasally, buccally, vaginally or via an implanted reservoir.

For use in a mammal, the cosmetic, pharmaceutical, or nutraceutical composition may be administered subcutaneously, intramuscularly, intramammarily, intrathecally, or intravenously.

For use in a mammal, the cosmetic, pharmaceutical, or nutraceutical composition may be administered topically or intradermal or transdermal. Liquid compositions may include ointments, creams, gels, lotions, aqueous liquids, which may be formulated inside a transdermal patch. Especially for skin treatment, the particles as defined herein may be dispersed or loaded in a hydrogel or a spray.

For agricultural use, the particles as defined herein may be formulated in a suspension or a paste or a reservoir adapted for administration to a plant or soil.

### Medical and Other Uses

The particles as defined herein, especially the cosmetic, pharmaceutical, agricultural or nutraceutical composition as defined above may be used as dietary supplement, food additive or for agricultural applications to plants or to ground/soil.

The invention thus relates to a method of treating a disease or disorder comprising administering the particle as defined herein, or the cosmetic, pharmaceutical, or nutraceutical composition as defined herein, to a subject in need thereof.

The invention thus relates to a method of treating, preventing or reducing the risk of a disorder, which comprises administering to a subject, in need thereof, a therapeutically effective amount of the particle as defined herein, or the pharmaceutical composition as defined herein. The invention thus relates to a method of cosmetic treating, preventing or reducing the risk of a cosmetic or nutraceutical disorder, which comprises administering to a subject, in need thereof, a cosmetical or nutraceutical effective amount of the particle as defined herein, or the cosmetic, nutraceutical composition as defined herein.

The disorder may be a disease or illness and the like.

The subject may a mammal, such as a human or animal, or a plant, such as trees, shrubs, herbs, grasses, ferns, and mosses. The subject may be soil or ground or the subject may be a mineral.

The particle as defined herein, or the pharmaceutical composition as defined herein may be used for delivering oxygen, carbon monoxide, carbon dioxide or air to a subject. The particles may also be uses for prevention and/or treatment of a disease in a mammal. as a blood substitute, for example as substitute for red blood cells. For delivering gases, such as oxygen or for use as a blood substitute, the particles as defined herein, may comprise PFC as the agent, which may be PFOB or perfluoro-15-crown-5-ether and further comprising a gas selected from the group comprising oxygen, air, carbon dioxide, and carbon monoxide, preferably oxygen. These particle may comprise or consist of cyclic polysaccharide, which may be a α-, β-, or γ-cyclodextrin comprising 6, 7 and 8 α-D(+)-glucopyranoside units, or β-cyclodextrin, and wherein R is poly(E caprolactone).

The particles may also be uses for prevention and/or treatment of a skin disorder. The skin disorder may be selected from the group comprising acne vulgaris, fungal infection, viral infection, bacterial infection, contact dermatitis, atopic dermatitis, allergic dermatitis, eczema, hives, burn, sunburn, ulcer, psoriasis, impetigo, skin cancer, melanoma, basal cell carcinoma, scabies, seborrheic dermatitis, ringworm, wart, shingles, vitiligo, hyperpigmentation, cold sore, actinic keratosis, xerosis, pityriasis roses, athlete's foot, scar, cicatrization, irritation, dryness, redness, wrinkles, wound, abrasion, laceration, ulcer, bruise, scratch, puncture and avulsion.

A topically-delivered oxygen-carrying the particle as defined herein, or the cosmetic, nutraceutical composition as defined herein are able to provide abundant oxygen to cells, and thus are suitable for uses or applications selected from medicine, cosmetics, skin care, skin regeneration, hair care, wound healing, agriculture, and nutraceutics, as well as other industrial applications related to the industries of chemistry, materials, food, and agriculture.

For use in skin treatment, the agent encapsulated in the particles may be an antioxidant, a sequestering agent, a chelating agent, a steroid, and an anti-coagulant. These particle may comprise or consist of cyclic polysaccharide, which may be a α-, β-, or γ-cyclodextrin comprising 6, 7 and 8 α-D(+)-glucopyranoside units, or β-cyclodextrin, and wherein R is poly(ε caprolactone).

The particles may comprise a diagnostic or sensing molecules and be used for diagnosis of a disease in a mammal, such as diagnosis of cancer, infections, inflammations, and the like.

The particles may comprise a diagnostic or sensing molecules and be used for diagnosis of a disease in a plant, such as of the presence of an insecticide or pesticide.

### EXAMPLES

In order that embodiments of the invention described herein may be more fully understood, the following examples are set forth. The examples described in this application are offered to illustrate the compounds, pharmaceutical compositions, cosmetic compositions, nutraceutical compositions, and methods provided herein and are not to be construed in any way as limiting their scope.

### Materials and Methods

Chemicals and solvents were purchased from commercial suppliers or were fabricated and purified by standard techniques. The following commercial reagents were used as purchased without further purification: ε-Caprolactone (99%, Alfa Aesar), β-cyclodextrin (98%, Acros Organic), 1,5,7-triazabicyclo[4.4.0]dec-5-ene (98%, Sigma-Aldrich), dimethyl sulfoxide (DMSO, anhydrous, 99.9%, Sigma-Aldrich), molecular sieves (MS, 3 Å, 8 to 12 mesh, ACROS Organics), ethyl acetate (99.9%, FCC, FG, Sigma-Aldrich), isopropyl alcohol (99.5%, Reagent ACS/USP Grade, Lab Alley), D-α-Tocopherol polyethylene glycol 1000 succinate (VETPGS, BioXtra, water-soluble vitamin E conjugate, Sigma-Aldrich, 57668), 1-bromoheptadecafluorooctane (PFOB, =99%, Sigma-Aldrich, 343862).

### Example 1—a General Procedure for the Catalytic Synthesis of the PCL-CD Material

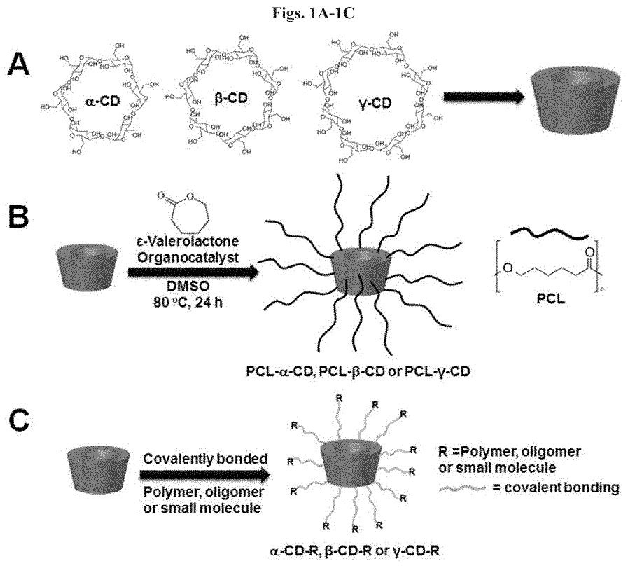

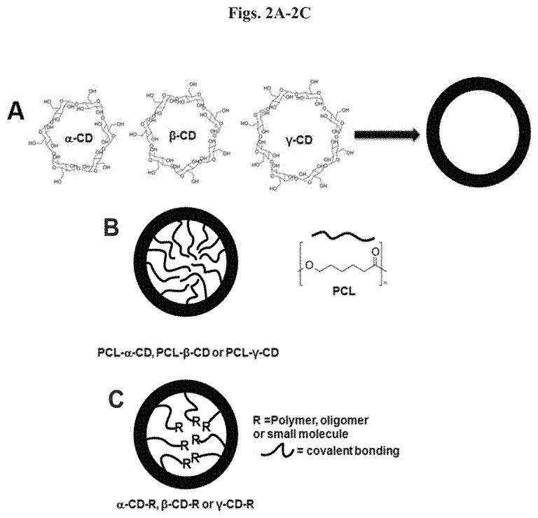

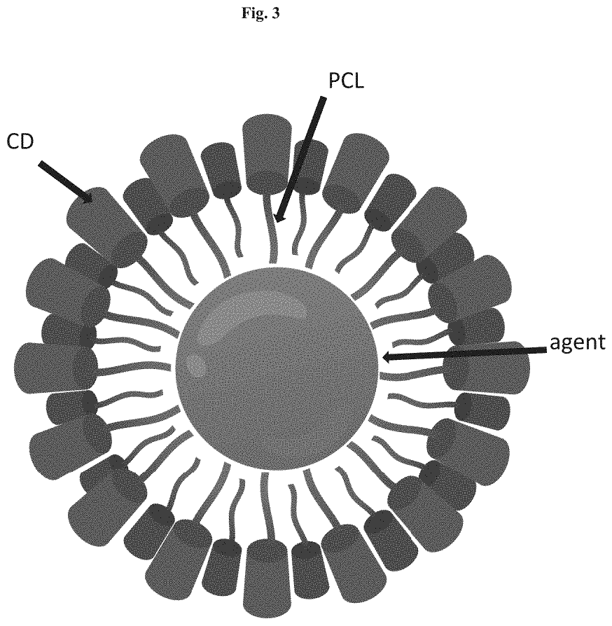

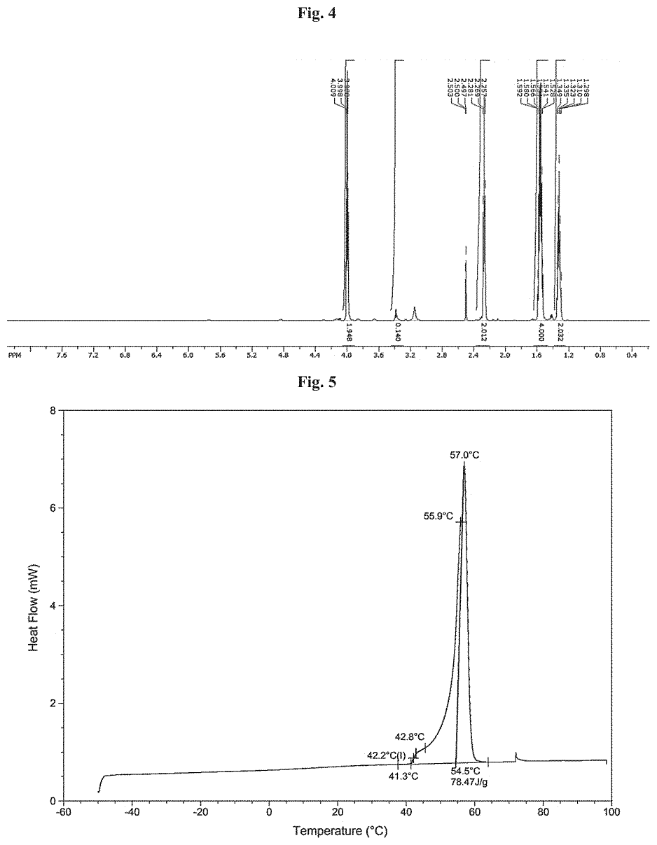

β-Cyclodextrin (16.67 g, 1.469 mmol, 1.0 equiv.) and TBD (8.33 g, 0.0598 mmol, 2.0 mol %) were dissolved in DMSO (500 g) in a dry glass flask. Subsequently, the solution was allowed to dry by sitting over MS (10.0 g) for 3 days at room temperature in a capped flask. In parallel, the ε-caprolactone was dried over MS for at least 3 days. After the drying, the solution was decanted into a dry flask and weight to calculate the exact amount of β-cyclodextrin in it. Afterwards, ε-caprolactone (333.4 g, 2.92 mol) was added. Next, the reaction was stirred at 80° C. for 24 h under a nitrogen atmosphere. After the completion of the reaction, the warm reaction mixture was transferred to 250-mL centrifuge tubes containing water in order to precipitate the polymer. After vortex and centrifugation, the supernatant was discarded, and the precipitated product was collected, rinsed with water (×3), and once with isopropanol. Any remaining solvent residue in the product was removed at 37° C. for 5 days under vacuum. The final product was obtained as a white solid in 80% isolated yield. See FIG. 1B for schematic representation of functionalization of β-cyclodextrin with ε-caprolactone-derived polyester. See FIG. 4 for the ¹H-NMR spectrum of the PCL-β-CD.

### Expanding the Scope of CD and Lactones:

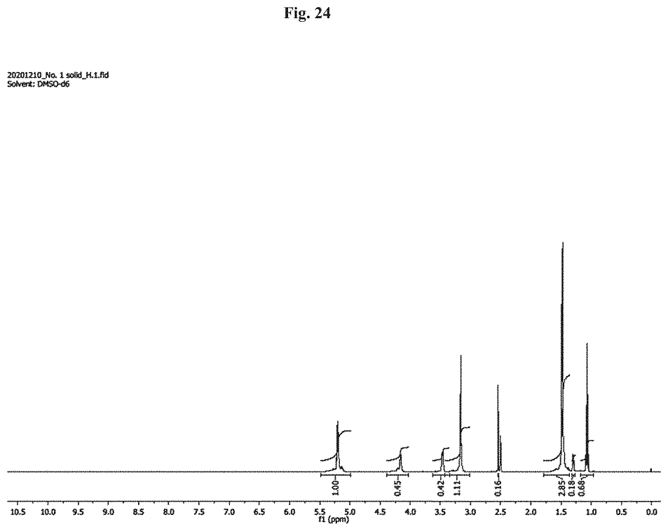

1. The α-cyclodextrin-L-lactide was fabricated following the described procedure in Example 1 with minor modifications (without molecular sieves, the reactions were performed at room temperature (about 20-25° C.) instead of 80° C., for 24 h, amount of catalyst 0.15 g and 9 g of DMSO, 0.3 g of α-cyclodextrin and 3 g of L-lactide were used. After the completion of the reaction, the mixture was rinsed with water once and with ethanol twice). Providing the compound in 61% yield. See FIG. 24.

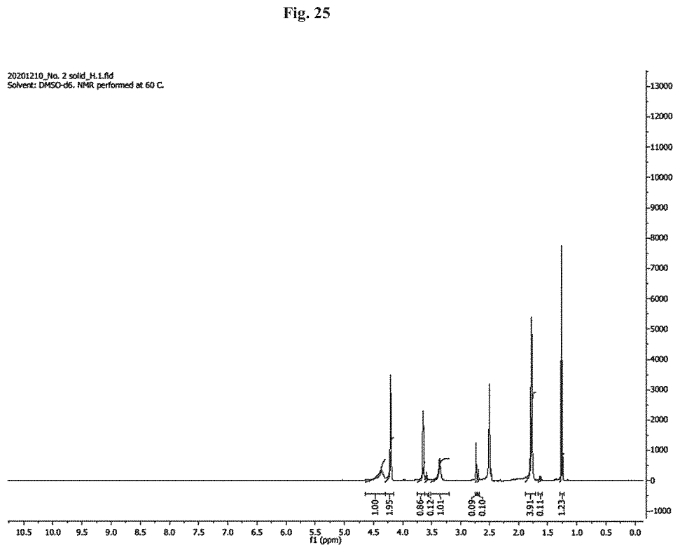

2. The α-cyclodextrin-δ-valerolactone was fabricated following the described procedure in Example 1 with minor modifications (without molecular sieves, the reactions were performed at room temperature (about 20-25° C.) instead of 80° C., for 24 h, amount of catalyst 0.15 g and 9 g of DMSO, 0.3 g of α-cyclodextrin and 6 g of δ-valerolactone were used. After the completion of the reaction, the mixture was rinsed with water once and with ethanol twice). Providing the compound in >95% yield (No purification was performed). See FIG. 25.

3. The β-cyclodextrin-L-lactide was fabricated following the described procedure in Example 1 with minor modifications (without molecular sieves, the reactions were performed at room temperature (about 20-25° C.) instead of 80° C., for 24 h, amount of catalyst 0.15 g and 9 g of DMSO, 0.3 g of β-cyclodextrin and 1.5 g of L-lactide were used). After the completion of the reaction, the mixture was rinsed with water once and with ethanol twice). Providing the compound in 39% yield. See FIG. 29.

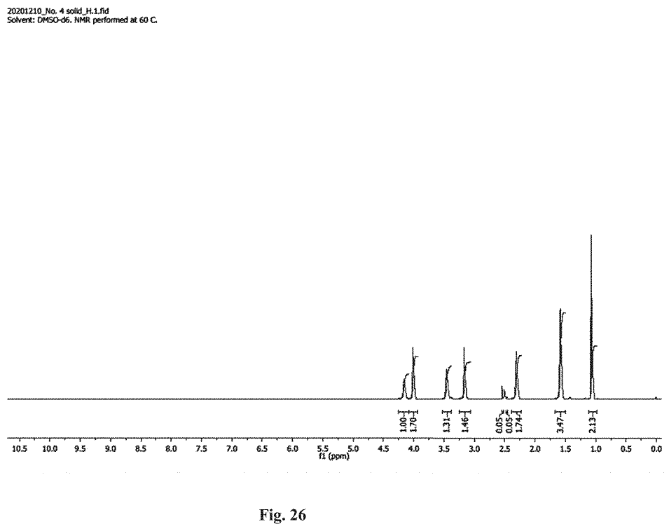

4. The β-cyclodextrin-δ-valerolactone was fabricated following the described procedure in Example 1 with minor modifications (without molecular sieves, the reactions were performed at room temperature (about 20-25° C.) instead of 80° C., for 24 h, amount of catalyst 0.15 g and 9 g of DMSO, 0.3 g of β-cyclodextrin and 6 g of δ-valerolactone were used. After the completion of the reaction, the mixture was rinsed with water once and with ethanol twice). Providing the compound in >95% yield (No purification was performed). See FIG. 26.

### Procedure for the ¹H NMR Analysis:

For the ¹H NMR analysis, the nanoparticle suspension was centrifuged at 3214 rcf for 30 min. The supernatant was decanted. DMF was added to the precipitate as the internal reference for ¹H NMR analysis and weighed the amount added. DMSO-d₆, was added and heated to 60° C. to dissolve the sample. The ¹H NMR was performed at 60° C. The encapsulated active ingredient was determined by comparing the peak area of such compound with the peak area of DMF peak.

### Example 2—Differential Scanning Calorimetry (DSC) Characterization of PCL-β-CD

DSC was conducted using a TA Instruments DSC 0100 calorimeter. The temperature started at −50° C. and went up at a rate of 5° C./min until reaching 100° C. A background control with no sample but an empty pan was run first. Then, 4.2 mg of PCL-β-CD was loaded in the same pan and was run under the same conditions. The DSC plot was presented after subtracting the background. See FIG. 5 for the DSC plot of the PCL-β-CD showing a glass transition temperature of 42.2° C. and a melting point around 57° C.

### Example 3—Microfluidic Formulation of PCL-β-CD/PFOB Micro/Nanoparticles

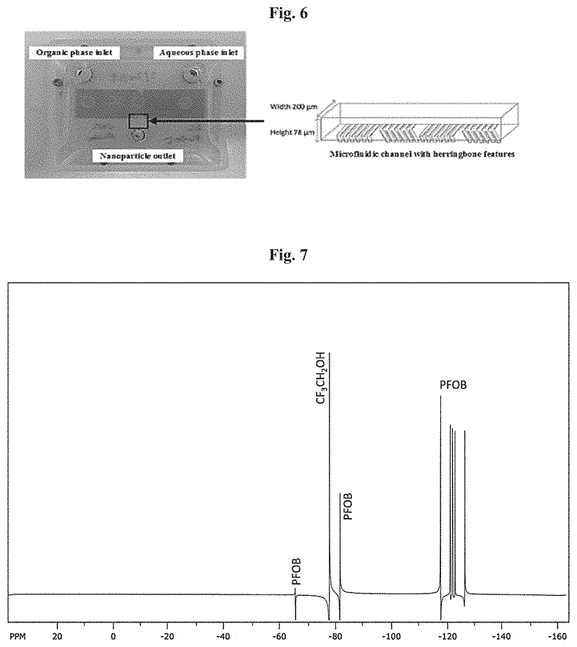

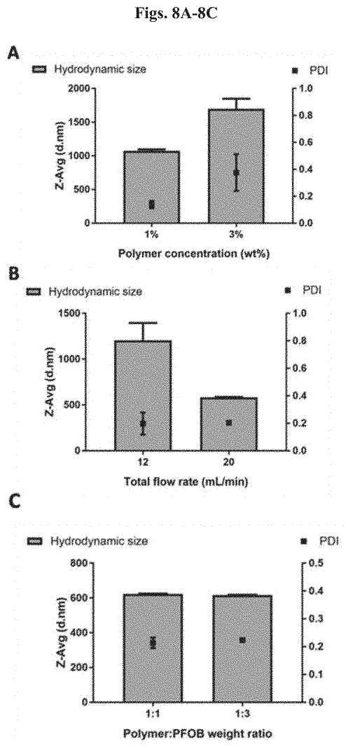

A microfluidic-based system (Nanoassemblr® reactor, Precision Nanosystems Inc., Vancouver, Canada) was used for micro/nanoparticle formulation. The polymer solution was prepared by dissolving 1% or 3% (w/v) PCL-β-CD and 3% or 9% (w/v) PFOB in ethyl acetate, and 1% (w/v) of VETPGS in deionized water solution was used as the surfactant. The organic phase (polymer solution) and the aqueous phase (surfactant solution) were mixed within a cartridge installed inside the reactor, which contains a microfluidic channel with 200 μm*79 μm (width*height) dimension with herringbone features. See FIG. 6 for a schematic representation of the cartridge with microfluidic channels employed in a Nanoassemblr® reactor. This mixing process was controlled by syringe pumps in terms of flow rate ratio (FRR) (ratio between organic and aqueous stream) as well as total flow rate (TFR). The FRR was set to 1:9, and the TFR was ranged from 12-20 mL/min. The formulated micro/nanoparticles were dialyzed at room temperature against DI water for one day to remove the residual organic solvent.

1 mL of the PCL-β-CD/PFOB particle dispersion in water was added to an Eppendorf tube and centrifuged at 100 RCF for 10 min. The supernatant was carefully decanted without losing any precipitate. Another 1 mL of the same dispersion was added to the same Eppendorf tube and centrifuged. The supernatant was carefully decanted. In order to remove the H₂O residue, 1 mL of D₂O was added to the same tube. It was gently shaken and centrifuged. Next, the supernatant was carefully decanted. This D₂O rinsing process was repeated once. Afterwards, 600 μL of ethyl acetate-d8 was added to dissolve the precipitate. Then, 15 μL of 2,2,2-trifluoroacetic acid was added as an internal reference. ¹⁹F-NMR was conducted on this sample. The amount of PFOB and the encapsulation efficiency was determined by the integration of the peaks of PFOB and 2,2,2-trifluoroacetic acid in the ¹⁹F-NMR spectrum. See FIG. 7 for the ¹⁹F-NMR spectrum of PCL-β-CD/PFOB dissolved in ethyl acetate-d₈ with 2,2,2-trifluoroacetic acid as an internal reference.

The formulated PCL-β-CD/PFOB micro/nanoparticles were characterized by transmission electron microscope (TEM, JEOL JEM-1010, Peabody, Mass.), scanning electron microscope (SEM, Hitachi S-4800, Japan), and dynamic light scattering (Zetasizer Pro, Malvern, United Kingdom).

### Example 4—the General Procedure for the Nanoparticle Fabrication Through Ultrasonication Approach

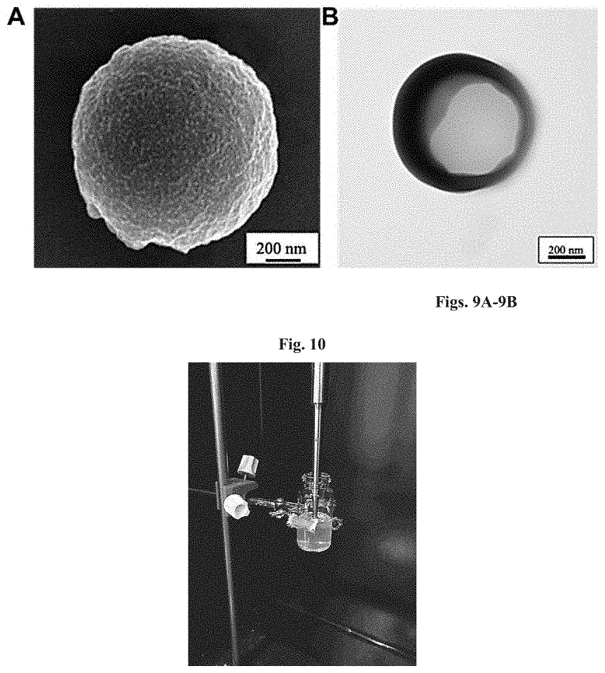

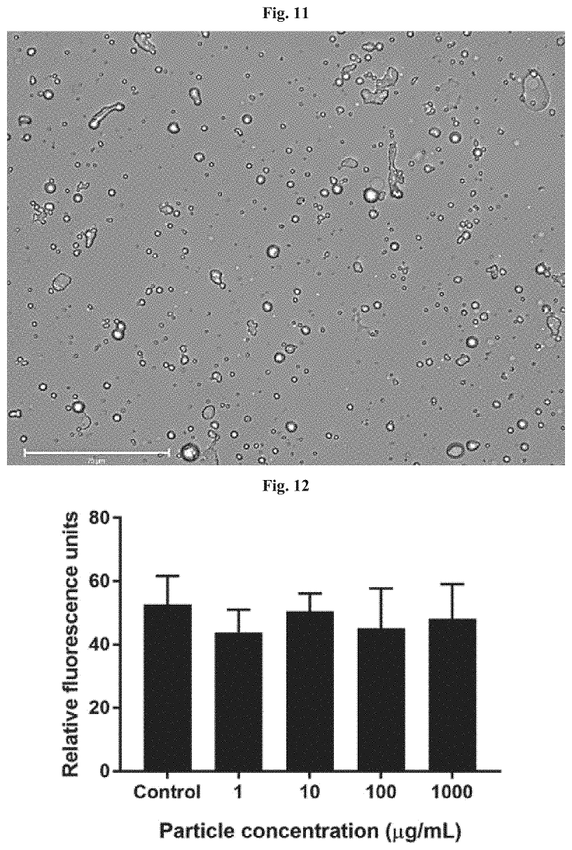

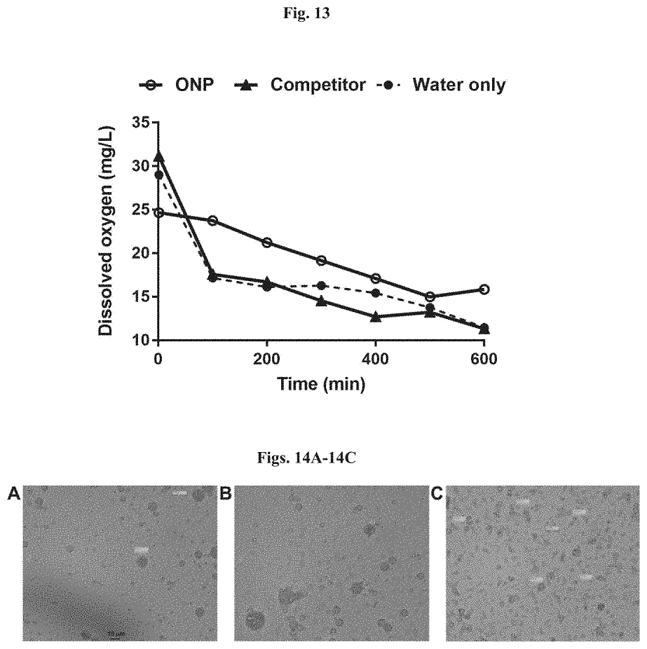

Probe sonication was employed to fabricate the micro/nanoparticles using an ultrasonic processor (Misonix Inc., NY, USA). The sonicator was operated at an amplitude of 20. In a 20 mL glass vial, 1 mL of polymer solution was added dropwise into a surfactant solution (10 mL) for 30 s (2 mL/mL). Afterwards, the reaction mixture was sonicated for additionally 30 s. See FIG. 10 for an image of the system employed for formulation of the PCL-β-CD/PFOB under sonication. The remaining residual organic solvent in the product was evaporated in a fume hood overnight at room temperature under stirring.

### Example 5—Microparticles Formulation by Solvent Evaporation

A laboratory or industrial high-speed mixer with a stainless-steel blade impeller was used to produce microparticles. The polymer solution (3% PCL-β-CD and 15% PFOB in ethyl acetate) was added slowly at 1.2 mL/min to 1% (w/v) VETPGS surfactant solution. The final volume ratio of the polymer solution to the surfactant solution was 1:9. The solution was mixed for another 30 s, and the residual organic solvent was evaporated at room temperature overnight under stirring. See FIG. 11 for the microscope images showing the morphology of PFOB-loaded PCL-β-CD particles.

### Example 6—Cytotoxicity Testing

The cytotoxicity of PCL-β-CD/PFOB particles was tested in vitro on NIH 3T3 fibroblast cells (Sigma-Aldrich 93061524). Cells were seeded in a 96-well plate (6,000 cells/cm²) and incubated for 24 h in Dulbecco's Modified Eagle Medium (DMEM) medium supplemented with 10% calf serum and 1% penicillin/streptomycin (P/S). Next, cells were treated with the particles suspended in the cell medium at the concentrations from 1 to 1,000 μg/mL for 48 h. Afterwards, the particles were removed, and the cells were rinsed with phosphate buffered saline (PBS). The cell density was determined by the PrestoBlue Cell Viability Reagent by measuring the fluorescence intensity (Ex=560 nm; Em=590 nm). Data were expressed as relative fluorescence unit (RFU) and the experiment was triplicated. See FIG. 12 for the bar graph showing the cell viability of 3T3 fibroblast cells after treatment with PFOB-loaded PCL-β-CD particles.

### Example 7—Dissolved Oxygen Measurement

The dissolved oxygen content in the PCL-β-CD/PFOB particle dispersion in water was compared to perfluorodecalin (PFD) dispersion and water alone. Briefly, 3 mL of the PCL-β-CD/PFOB particles of Example 3 (with 1.5% PFOB encapsulated) dispersion was saturated with pure oxygen (medical grade) for 30 min under mixing at 500 rpm. As the comparison groups, 1.5% PFD dispersed in water (competitor group) and water alone were saturated with oxygen by the same method. The dissolved oxygen of each sample was then measured by an oxygen sensor (NUL-205, Neulog, USA) over 10 h at 30° C. (surface temperature of the skin).

### Example 8—Preparation of PCL-CD/PFOB Microparticles

Reagents amount: PCL-CD (0.5563 g)+PFOB (1.62 g)+AcOEt (6 g)+H₂O (60 g)+VETPGS (0.6 g); Total weight with solvents=68.7763 g, Total weight without solvents=62.7763 g, Solid contents=2.7763 g; % Solids in emulsion=4.42 g, % PCL-CD (β-Cyclodextrin) in emulsion=0.89 g, % VETPGS in emulsion=0.96 g; % active in solids=58.35 g, % active in emulsion=2.58 g.

### Experimental Procedure:

Preparation of Polymer Solution with PFOB:

The PCL-CD (90 mg) was dissolved in 1 mL of ethyl acetate and the solution was subsequently sonicated at 40° C. for 15 min, providing a clear a completely dissolved polymer solution and free of precipitate. Afterwards PFOB (270 mg) was slowly added (about 3 μL PFOB was added each 20 seconds) to the 1 mL polymer solution under gently stirring at 40° C. The reaction mixture appeared to be clear and free of precipitates.

### Preparation of Surfactant Solution:

The Vitamin E TPGS 1000 (VETPGS) (1 g) was dissolved in 100 mL of water by vigorous stirring for 15 min.

### Emulsion Process:

10 mL of the surfactant solution of VETPGS was added to a 100 mL reactor and subsequently stirring at 400 rpm at 40° C. Next, 1000 μL of the polymer solution with PFOB (organic phase) was added in about 5×200-250 μL each 7 seconds with a total time of 35 seconds. The white solution turned completely clear. Afterwards the solution was mixed for 5 minutes at 400 rpm at 40° C. providing spherically microparticles of about 10-25 μm observed by optical microscopy (OM). See FIGS. 14A-14B. The solution was mixed for additional 16 minutes at 400 rpm at 40° C. providing spherically microparticles of about 1-10 μm. See FIG. 14C.

### Final Purification Steps (Two Options):

### Option 1—Evaporation:

The residual organic solvent in the reaction mixture was evaporated in a fume hood overnight (about 12-24 h) at room temperature under (about 24° C.) stirring.

### Option 1—Dialysis in MilliQ H₂O:

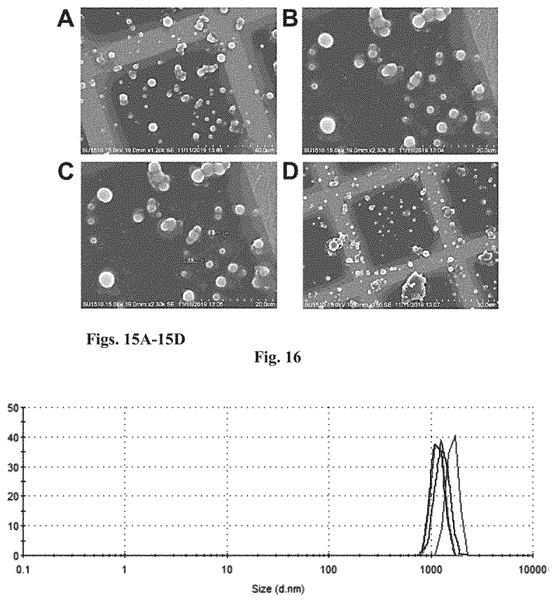

The microparticles were dialyzed for 48 h with MilliQ water at room temperature (about 24° C.) with dialysis membranes of 12-14 kDa. See FIGS. 15A-15D.

### Example 9—Preparation of PCL-CD/Vitamin A-Propionate Microparticles

Reagents amount: PCL-CD (0.5563 g)+Vitamin A-propionate (1.62 g)+AcOEt (6 g)+H₂O (60 g)+VETPGS (0.6 g); Total weight with solvents=68.7763 g, Total weight without solvents=62.7763 g, Solid contents=2.7763 g; % Solids in emulsion=4.42 g, % PCL-CD in emulsion=0.89 g, % VETPGS in emulsion=0.96 g; % active in solids=58.35 g, % active in emulsion=2.58 g.

### Experimental Procedure:

Preparation of Polymer Solution with Retinyl Propionate:

The PCL-CD (90 mg) was dissolved in 1 mL of ethyl acetate and the solution was subsequently sonicated at 40° C. for 15 min, providing a clear a completely dissolved polymer solution and free of precipitate. Afterwards retinyl propionate (81 mg) was slowly added (about 3 μL retinyl propionate was added each 20 seconds) to the 1 mL polymer solution under gently stirring at 40° C. The reaction mixture appeared to be clear and free of precipitates.

### Preparation of Surfactant Solution:

The Vitamin E TPGS 1000 (VETPGS) (1 g) was dissolved in 100 mL of water by vigorous stirring for 15 min.

### Emulsion Process:

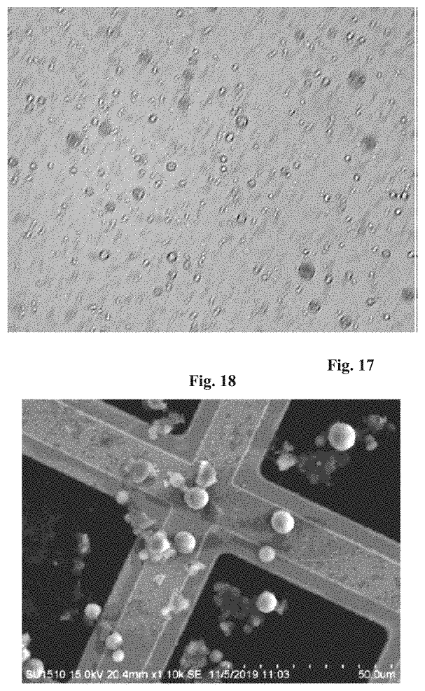

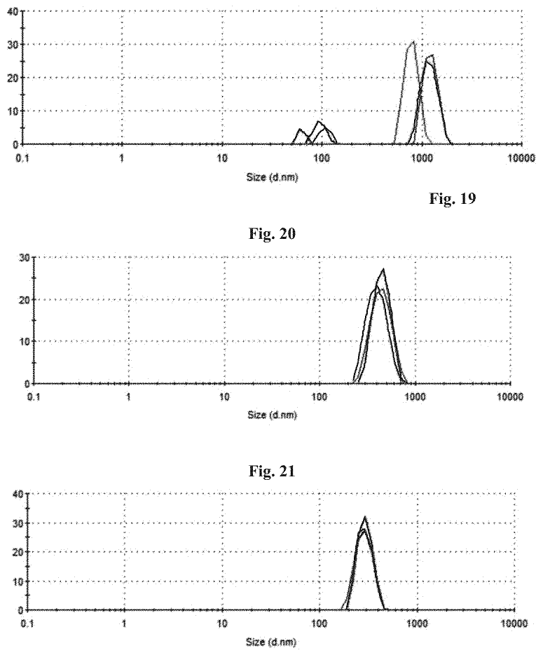

10 mL of the surfactant solution of VETPGS was added to a 100 mL reactor and subsequently stirring at 400 rpm at 40° C. Next, 1000 μL of the polymer solution with retinyl propionate (organic phase) was added in about 5×200-250 μL each 7 seconds with a total time of 35 seconds. The white solution turned completely clear. Afterwards the solution was mixed for 20 minutes at 400 rpm at 40° C. providing spherically microparticles of about 1 μm observed by optical microscopy (OM). See FIGS. 17-19.

### Final Purification Steps (Two Options):

### Option 1—Evaporation:

The residual organic solvent in the reaction mixture was evaporated in a fume hood overnight (about 12-24 h) at room temperature (about 24° C.) under stirring.

### Option 1—Dialysis in MilliQ H₂O:

The microparticles were dialyzed for 48 h with MilliQ water at room temperature (about 24° C.) with dialysis membranes of 12-14 kDa.

### Example 10—Preparation of PCL-CD/PFOB Nanoparticles

Reagents amount: PCL-CD (0.1 g)+PFOB (0.27 g)+Amphiphilic polymer (0.8314 g)+THF (1 g)+H₂O phase inv (10 g)+AcOEt (0.75 g)+VETPGS (0.1 g); Total weight with solvents=12.9514 g, Total weight without solvents=10.9325 g, Solid contents=0.9325 g; % Solids in emulsion=8.53 g, % PCL-CD in emulsion=0.915 g, % VETPGS in emulsion=0.92 g; % active in solids=28.95 g, % active in emulsion=2.47 g.

### Experimental Procedure:

### Co-Solubilization of Organic Phase:

To a 3 mL glass vial was added PCL-CD (0.1000 g), VETPGS (0.1000 g), neutral amphiphilic polymer (0.8314 g) (altering the amount of this components will modify the size of the nanoparticles), THF (0.6700 g) (1 g can also be added but the 0.67 g provided enough solubilization). Subsequently the reaction mixture was mixed with magnetic stirring and ultrasonication in order to obtain a clear and transparent solution. Afterwards, PFOB (0.27 g) was added under magnetic stirring. The solution turned turbid due to the poor solubility of PFOB in THF, therefore AcOEt (075 g) was added dropwise providing a transparent solution.

### Emulsion Process:

The organic phase was added into a 3-neck flask equipped with mechanical stirring. To this mixture MilliQ (10 g) water was added dropwise using an additional funnel at 350 rpm over 5 minutes.

### Final Purification:

### Dialysis in MilliQ H₂O:

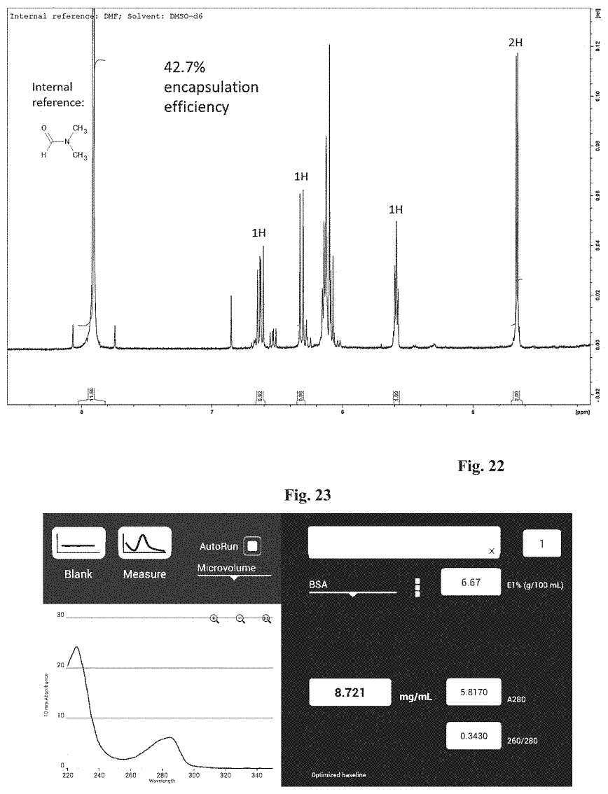

The nanoparticles were dialyzed for 48 h with MilliQ water at room temperature (about 24° C.) with dialysis membranes of 12-14 kDa. See FIGS. 20-21.

### Example 11—Preparation of PCL-CD/Vitamin A-Propionate Nanoparticles

Reagents amount: PCL-CD (0.1 g)+Vitamin A-propionate (0.27 g)+Amphiphilic polymer (0.8314 g)+THF (1 g)+H₂O phase inv (10 g)+VETPGS (0.1 g); Total weight with solvents=12.2014 g, Total weight without solvents=10.9325 g, Solid contents=0.9325 g; % Solids in emulsion=8.53 g, % PCL-CD in emulsion=0.915 g, % VETPGS in emulsion=0.92 g; % active in solids=28.95 g, % active in emulsion=2.47 g.

### Experimental Procedure:

### Co-Solubilization of Organic Phase:

To a 3 mL glass vial was added PCL-CD (0.1000 g), VETPGS (0.1000 g), neutral amphiphilic polymer (0.8314 g) (altering the amount of this components will modify the size of the nanoparticles), THF (0.6700 g) (1 g can also be added but the 0.67 g provided enough solubilization), Vitamin A-propionate (retinyl propionate) (0.27 g). Subsequently the reaction mixture was mixed with magnetic stirring and ultrasonication in order to obtain a clear and transparent solution.

### Emulsion Process:

The organic phase was added into a 3-neck flask equipped with mechanical stirring. To this mixture MilliQ (10 g) water was added dropwise using an additional funnel at 350 rpm over 2 minutes.

### Final Purification:

### Dialysis in MilliQ H₂O:

The nanoparticles were dialyzed for 48 h with MilliQ water at room temperature (about 24° C.) with dialysis membranes of 12-14 kDa. See FIGS. 20-21.

The encapsulation efficiency was calculated by using ¹H NMR analysis confirming 42.7% encapsulation efficiency of the vitamin A-propionate within the nanoparticle delivery cargo. See FIG. 22.

### Sample Preparation for the SEM Characterization:

A 10 L of each solution of sample was deposited onto TEM grid, followed by letting it sit for 1 min. The excess of solvent from the edge was removed by a filter paper, and the grid sample was air dried overnight before characterization. The next day, the TEM grid was put onto a double-sided carbon tape mounted on a SEM support. In addition, all the samples were sputter coated using gold (90 seg/20 mA). This process was carried out in the same way for all of concentrations.

Sample Preparation for the DLS Characterization: The samples for the DLS analysis was diluted to 0.05% in solids.

### Summary

| | | | | | Volume ratio | | |
|---|---|---|---|---|---|---|---|
| | | | | | (polymer to | | |
| Particle | | | Stirring | OM/SEM/ | surfactant | Purification | |
| size | Emulsifier | Method | (rpm) | DLS | solution) | methods | Results |
| Micro | VETPGS | Oil onto | 400 | 1-10 μm | 1:10 | Evaporation | Microparticles |
| | | Water | | spherical | | (r.t., fume hood | were obtained |
| | | | | morphology | | overnight) | for both |
| | | | | | | Dialysis in | formulations |
| | | | | | | MilliQ H₂O | (PFOB and |
| | | | | | | | Vitamin A- |
| | | | | | | | propionate). |
| | | | | | | | Procedures |
| | | | | | | | were carried |
| | | | | | | | out by taking |
| | | | | | | | into account |
| | | | | | | | the scale |
| | | | | | | | up process |
| Nano | VETPGS | Oil onto | 400- | — | 1:10 | — | Nanoparticles |
| | | Water and | 600 | | | | were not |
| | | Water | | | | | obtained |
| | | onto Ooil | | | | | for neither |
| | | co- | | | | | formulation |
| | | solvents | | | | | (PFOB or |
| | | | | | | | Vitamin A- |
| | | | | | | | propionate). |
| | | | | | | | (Fast stirring |
| | | | | | | | and O/W) |
| Nano | VETPGS + | W onto O | 350 | 300-400 nm | 1:10 | Evaporation | Nanoparticles |
| | Amphiphilic- | (Self- | | spherical | | (r.t., fume hood | were obtained |
| | polymer | emulsion | | morphology | | overnight) | for both |
| | | technology) | | | | Dialysis in | formulations |
| | | | | | | MilliQ H₂O | (PFOB and |
| | | | | | | | Vitamin A- |
| | | | | | | | propionate). |
| | | | | | | | (Fast stirring |
| | | | | | | | and O/W, not |
| | | | | | | | modified |
| | | | | | | | % active). |
| | | | | | | | The PCL-CD/ |
| | | | | | | | Vitamin A- |
| | | | | | | | propionate |
| | | | | | | | remains |
| | | | | | | | stable one |
| | | | | | | | week later. |

### Certificate of Analysis: PCL-CD/PFOB Nanoparticles

| PROPERTIES | METHODS | RESULTS | SPECIFICATIONS |
|---|---|---|---|
| Color | IL-60 | Conforms | Pale white |
| Appearance | IL-60 | Conforms | Fluid dispersion |
| Odor | Sensory | Conforms | Characteristic |
| Spectrum FT-IR-ATR | IL-59 | Conforms | Absence NCO |
| | | | band |
| Solids | IL-07 | 8.13% | |
| Particle Size | IL-50, IL-11 | 270.2 ± 0.1 nm | |
| pH | IL-41 | 7.88 | |

### Certificate of Analysis: PCL-CD/Vitamin A-propionate Nanoparticles

| PROPERTIES | METHODS | RESULTS | SPECIFICATIONS |
|---|---|---|---|
| Color | IL-60 | Conforms | Pale white |
| Appearance | IL-60 | Conforms | Fluid dispersion |
| Odor | Sensory | Conforms | Characteristic |
| Spectrum FT-IR-ATR | IL-59 | Conforms | Absence NCO |
| | | | band |
| Solids | IL-07 | 6.11% | |
| Particle Size | IL-50, IL-11 | 354.9 ± 1.6 nm | |
| pH | IL-41 | 6.57 | |

### Example 12—Procedure for the Determination of Loading Efficacy of Proteins in the Nanoparticle Delivery System

The nanoparticles were manufactured, and the resulting supernatant was saved to analyze the amount of protein that did not load inside the nanoparticles. A concentration of 10 mg/mL of Bovine Serum Albumin (BSA) protein was used during the nanoparticle fabrication.

After analysis of the supernatant using Nanodrop spectrophotometer, it was detected that 8.721 mg/mL of BSA was presented in the supernatant. Considering that the total amount of protein that was used during the fabrication was 10 mg/mL and 8.721 mg/mL were present in the supernatant, it can be deduced that 1.279 mg/mL of BSA were successfully loaded inside the nanoparticles (12.79% of encapsulation efficiency) (an expected number since BSA is hydrophilic, and polymer nanoparticle core is hydrophobic). See FIG. 23.

### Example 13—the Release Study of Various Agents from the Nanoparticle Delivery System

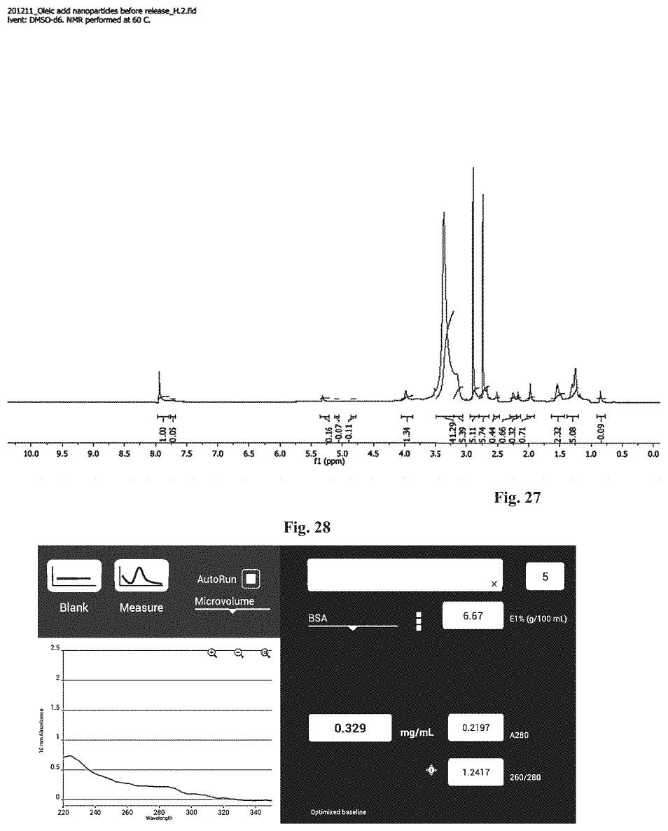

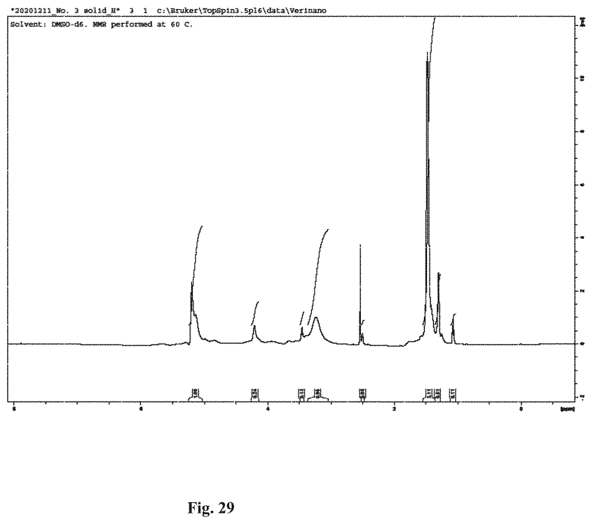

Nanoparticles encapsulating rapamycin, oleic acid and BSA protein were manufactured separately and incubated individually in PBS in a shaking device for 9 hours at room temperature and molecular release from the nanoparticles was quantified. To determine the release of rapamycin and oleic acid from the nanoparticles, DMSO-d₆ was used to break the nanoparticles and NMR to quantify the total amount of rapamycin and oleic acid left inside them after 9 hours of incubation in PBS at room temperature (rapamycin and oleic acid were encapsulated separate synthesis, not in the same nanoparticle). BSA Protein release from the nanoparticles was quantified via nanodrop spectrophotometer to determine the concentration of protein in the supernatant, after creating a nanoparticle pellet on centrifugation, all these after 9 hours of incubation in PBS at room temperature. The release was quantified after 9 h. The release was quantified after 9 hours of incubation in PBS at room temperature a concentration of 0.329 mg/ml of BSA protein was detected in the supernatant of the nanoparticles. Based on the encapsulation efficacy data shown before where 1.279 mg/ml of BSA protein were encapsulated inside the nanoparticles, the 0.329 mg/ml concentration of BSA protein in the supernatant represents a 25.72% release of the total protein content encapsulated in the nanoparticles. See FIG. 28. The encapsulation efficiency of oleic acid was 19% as determined by 1H-NMR. See FIG. 27.

### Pharmaceutical Formulation

| | | | | batch |
|---|---|---|---|---|
| | | | | size |
| | | | % by | 500.00 |
| phase | function | Ingredient | weight | gram |
| a | diluent | water (aqua) | 57.50 | 287.50 |
| a | humectant | glycerin | 2.00 | 10.00 |
| a | thickener | xanthan gum | 0.50 | 2.50 |
| b | humectant/solvent | propanediol | 3.00 | 15.00 |
| b | preservative | caprylyl glycol | 1.00 | 5.00 |
| | | ethylhexylglycerin | | |
| b | preservative | benzyl alcohol | 0.60 | 3.00 |
| | | ethylhexylglycerin | | |
| | | tocopherol | | |
| c | antioxidant | pentaerythrityl tetra-di- | 0.20 | 1.00 |
| | | t-butyl | | |
| | | hydroxyhydrocinnamate | | |
| c | emulsifier | cetearyl olivate | 3.00 | 15.00 |
| | | sorbitan olivate | | |
| c | emulsifier | glyceryl stearate | 1.50 | 7.50 |
| c | emulsion stabilizer | cetearyl alcohol | 0.50 | 2.50 |
| c | emulsifier | sorbitan stearate | 0.20 | 1.00 |
| c | emollient | coco-caprylate/caprate | 5.00 | 25.00 |
| c | emollient | dicaprylyl carbonate | 5.00 | 25.00 |
| c | emollient | squalane | 2.00 | 10.00 |
| c | thickeners | hydroxyethyl | 1.00 | 5.00 |
| | | acrylate/sodium | | |
| | | acryloyldimethyl taurate | | |
| | | copolymer | | |
| c | antioxidant | tocopheryl acetate | 1.00 | 5.00 |
| d | active ingredient | glycoproteins (and) | 3.00 | 15.00 |
| | | glutamic acid (and) | | |
| | | valine (and) threonine | | |
| | | (and) aqua (and) | | |
| | | phenoxyethanol (and) | | |
| | | ethyl hexylglycerin (and) | | |
| | | sodium metabisulfite | | |
| d | active ingredient | resveratrol polymethyl | 2.00 | 10.00 |
| | | methacrylate | | |
| | | tricaprylin | | |
| d | active ingredient | PCL-β-CD | 1.00 | 5.00 |
| d | active ingredient | pearl powder | 1.00 | 5.00 |
| d | active ingredient | avena sativa kernal | 1.50 | 7.50 |
| | | flower | | |
| e | humectant | sodium hyaluronate | 2.00 | 10.00 |
| | | water phenoxyethanol | | |
| el | humectant | hydrolyzed hyaluronic | 0.50 | 2.50 |
| | | acid | | |
| el | diluent | water (aqua) | 5.00 | 25.00 |
| f | ph adjuster | sodium hydroxide | 0.00 | 0.00 |
| | | water | | |
| f | phadjuster | water citric acid | 0.00 | 0.00 |
| | | | 100.00 | 500.00 |

### Phase a

Mix xanthan gum in glycerin until uniformly dispersed. Start heating water to 70-75° C. with mixing and slowly add xanthan gum/glycerin mixture

### Phase b

Mix propanediol, caprylyl glycol, ethylhexylglycerin, benzyl alcohol, ethylhexylglycerin and tocopherol together (heat if necessary to 50-55° C. to insure uniformity). Add phase b to phase a and maintain temperature at 70-75° C.

### Phase c

Mix pentaerythrityl tetra-di-t-butyl hydroxyhydrocinnamate, cetearyl olivate, sorbitan olivate, glyceryl stearate, cetearyl alcohol, sorbitan stearate, coco-caprylate/caprate, dicaprylyl carbonate, squalane, hydroxyethyl acrylate/sodium acryloyldimethyl taurate copolymer, tocopheryl acetate, together and heat with mixing to 70-75° C.

When uniform and all solids have melted, slowly add phase c to phase a/b.

Homogenize for 10 minutes at 4000 rpms at 70-75° C.

Switch to paddle mixing and start cooling to 40-45° C. slowly with mixing

### Phase d

Add Glycoproteins (and) Glutamic Acid (and) Valine (and) Threonine (and) Aqua (and) Phenoxyethanol (and) Ethylhexylglycerin (and) Sodium metabisulfite, resveratrol, polymethyl methacrylate, tricaprylin, PCL-β-CD, pearl powder, Avena sativa kernal flower (OAT COM IRR®), one at a time and mix with phase c until each ingredient dissolves or is thoroughly dispersed.

### Phase e

Add sodium hyaluronate, water, phenoxyethanol, and mix until uniform.

### Phase e1

Mix hydrolyzed hyaluronic acid, water (aqua) and when powder dissolves add phase e1 to batch mix until batch is uniform

### Phase f

Adjust pH with phase f to 5.5-6.0

The present invention is not limited to the embodiments disclosed but may be varied and modified within the scope of the following claims. For example, the particles may be made of a variety of cyclic polysaccharide combined with a variety of R radicals tuned for the encapsulation of a number of agents and drugs.

1. A cyclic polysaccharide compound having structural formula (I): 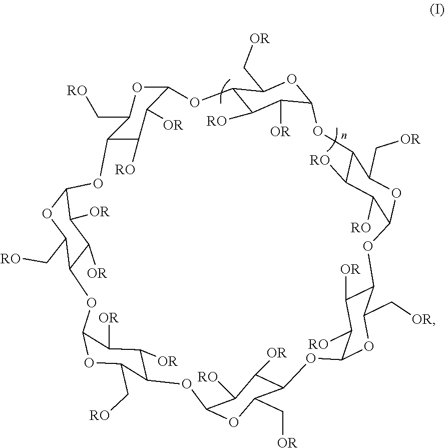
wherein
n is 0, 1, or 2; and,
R is, independently for each occurrence, H or a radical of polyester, polyethylene glycol, poly(anhydride), polyamide, polyorthoester, poly(L-lactide), poly(D-lactide), poly(D,L-lactide), polyethyleneimine, and co-polymer thereof, an oligomer, a protein, a peptide, an antibody, a cell receptor targeting ligand, a fatty acid, a lipid, phenol, a cinnamic acid, a quaternary ammonium group, an amino acid, or co-polymer thereof, provided at least one instance of R is not H.

2. The cyclic polysaccharide compound according to claim 1, wherein the cyclic polysaccharide is a α-, β-, or γ-cyclodextrin comprising 6, 7 and 8 α-D(+)-glucopyranoside units, or a mixture thereof.

3. The cyclic polysaccharide compound according to claim 1, wherein R is a radical of structural formula (II): 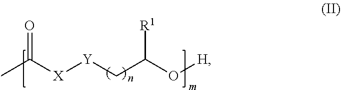
wherein
n is 0 to 3,
m is 2 to 300,000,
X is absent or selected from —CH₂—, —C(H)(OH)—, —O—, —N(H)—, or —C(H)(Hal)-,
Y is absent or selected from —CH₂—, —C(H)(OH)—, —O—, —N(H)—, or —C(H)(Hal)-,
R¹ is H, —OH, alkyl, aryl, or alkenyl, and
Hal is Cl, Br, or I.

4. The cyclic polysaccharide compound according to claim 1, wherein R is a polylactone selected from the group comprising caprolactone, valerolactone, glycolide, lactide, ethylglycolide, hexylglycolide and isobutylglycolide, or mixtures thereof.

5. The cyclic polysaccharide compound according to claim 2, wherein the cyclic polysaccharide is a α- or β-cyclodextrin comprising 6, 7 and 8 α-D(+)-glucopyranoside units, or a mixture thereof and R is caprolactone, valerolactone, glycolide or lactide.

6. A particle comprising a plurality of cyclic polysaccharide compounds according to claim 1, and one or more agent, wherein the plurality of said cyclic polysaccharide compounds form a hollow sphere, and the one or more agent is encapsulated within the hollow sphere; and
wherein the one or more agent is non-covalently associated with the cyclic polysaccharide compound, optionally further comprising a surfactant.

7. The particles according to claim 6, wherein the one or more agent is selected from a group comprising
perfluorocarbon (PFC) selected from the group comprising perfluorooctyl bromide (PFOB), perfluoro(tert-butylcyclohexane), perfluorodecalin (PFD), perfluoroisopropyldecalin, perfluoro-tripropylamine, perfluorotributylamine, perfluoro-methylcyclohexylpiperidine, perfluoro-octylbromide, perfluoro-decylbromide, perfluoro-dichlorooctane, perfluorohexane, dodecafluoropentane, and perfluoro crown ether,
a vitamin selected from the group comprising retinol (vitamin A), vitamin A-propionate, thiamine (vitamin B1), riboflavin (vitamin B2), niacin (vitamin B3), pantothenic acid (vitamin B5), pyridoxine (vitamin B6), biotin (vitamin B7), folic acid (vitamin B9), ascorbic acid (vitamin C), ergocalciferol (vitamin D1), and tocopherols (vitamin E),
a protein selected from the group comprising an enzyme, an antibody, a CAS protein, a transmembrane protein, an amino acid, a cell signaling proteins, and a structural protein such as collagen, hyaluronan, elastin, and tropoelastin,
a fatty acid selected from the group comprising essential, saturated, non-saturated, short chain, medium chain, long chain, very long chain fatty acids, selected but not limited to caprylic acid, capric acid, lauric acid, myristic acid, palmitic acid, stearic acid, arachidic acid, behenic acid, lignoceric acid, cerotic acid, palmitoleic acid, oleic acid, myristoleic acid, linoleic acid, sapienic acid, elaidic acid, vaccenic acid, linoelaidic acid, α-linolenic acid, erucic acid, docosahexaenoic acid, eicosapentaenoic acid, and arachidonic acid.
an imaging agent selected from the group comprising diagnostic imaging agent, a sensing molecule, a contrast agent, a fluorescence sensor, an electrochemical sensor, an electronic sensor, a peptide, an aptamer, a quantum dot, a metallic particle, and a radioisotope of a drug,
genetic material,
insecticide and a pesticide, and
a drug, or any combination thereof.

8. The particles according to claim 6, wherein the one or more agent is selected from a group comprising vitamins or vitamin A, vitamin A-propionate, vitamin E, rapamycin, oleic acid and BSA protein, perfluorocarbon (PFC) or perfluorooctyl bromide (PFOB) or perfluoro crown ether.

9. The particle according to claim 6, wherein the surfactant is a non-ionic surfactant selected from the group comprising D-α-Tocopherol polyethylene glycol 1000 succinate (VETPGS), poly(vinyl acetate) (PVA), TWEEN®20, TWEEN®40, TWEEN®80, POLYSORBATE®20, POE (4) hydrogenated castor oil, and BRIJ®96.

10. The particle according to claim 6, wherein the cyclic polysaccharide is a α- or β-cyclodextrin comprising 6, 7 and 8 α-D(+)-glucopyranoside units, or a mixture thereof and R is caprolactone, valerolactone or lactides and the surfactant is VETPGS.

11. The particle according to claim 7, further comprising one or more addition drug, and wherein the one or more additional drug is selected from a group comprising
antibiotic drug selected from a group comprising penicillins such as penicillin, penicillin G, hetacillin potassium, cloxacillin benzathine, ampicillin and amoxicillin trihydrate, aminocoumarins such as novobiocin, cephalosporins such as cephalexin, ceftiofur sodium, ceftiofur hydrochloride, ceftiofur crystalline free acid, macrolides such as tildipirosin, tylosin, tulathromycin, erythromycin, clarithromycin, and azithromycin, quinolones and fluoroquinolones such as enrofloxacin, ciprofloxacin, levofloxacin, and ofloxacin, sulfonamides such as sulfadimethoxine, co-trimoxazole and trimethoprim, tetracyclines such as tetracycline, oxytetracycline and doxycycline, aminoglycosides such as dihydrostreptomycin sulfate, neomycin, gentamicin and tobramycin, lincosamides such as pirlimycin hydrochloride, lincomycin, clindamycin, and pirlimycin, and amphenicols such as florfenicol,
an antiparasitic drug selected from a group comprising antiprotozoals such as melarsoprol, eflornithine, metronidazole, tinidazole, miltefosine, antihelminthics such as mebendazole, pyrantel pamoate, thiabendazole, diethylcarbamazine, ivermectin, aticestodes such as niclosamide, praziquantel, albendazole, antitrematodes such as praziquantel, antiamoebics such as rifampin and amphotericin B, and broad-spectrum drugs such as nitazoxanide,
an antimycotic drug selected from a group comprising polyenes such as amphotericin b, candicidin, filipin, hamycin, natamycin, nystatin, and rimocidin; azoles such as imidazole, triazole, thiazole, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, and abafungin; allylamines such as amorolfin, butenafine, naftifine, and terbinafine; echinocandins such as anidulafungin, caspofungin and micafungin; and others such as aurones, benzoic acid, ciclopirox olamine, flucytosine or 5-fluorocytosine, griseofulvin, haloprogin, tolnaftate, undecylenic acid, triacetin, crystal violet, castellani's paint, orotomide (f901318), miltefosine, potassium iodide, coal tar, copper(ii) sulfate, selenium disulfide, sodium thiosulfate, piroctone olamine, iodoquinol, clioquinol, acrisorcin, zinc pyrithione, and sulfur,
a coloring agent or dye selected from a group comprising Quinoline yellow, Ponceau 4R, Carmoisine, Patent Blue V, Greens S, Brilliant Blue FCF, Indigotine, Fast Green FCF, Erythrosine, Sunset Yellow, Allura Red AC, Tartrazine, Sunset Yellow FCF, Spirulina, and Betanin,
analgesic or anti-inflammatory drug selected from a group comprising aspirin, ibuprofen, and naproxen, naproxen sodium, diclofenac, acetoaminophen, celecoxib, piroxicam, indomethacin, meloxicam, ketiprofen, sulindac, diflunisal, nabumetone, oxaprozin, tolmetin, salsalate, etodolac, fenoprofen, flurbiprofen, ketorolac, meclofenamate, and mefenamic acid,
a corticosteroid selected from a group comprising prednisone, betamethasone, cortisone, dexamethasone, hydrocortisone, methylprednisolone, prednisolone and triamcinolone acetonide.
an anti-acid drug selected from a group comprising nizatidine, famotidine, cimetidine, ranitidine, omeprazole, esomeprazole, lansoprazole and sodium bicarbonate,
a diuretic selected from a group comprising chlorthalidone, chlorothiazide, hydrochlorothiazide, indapamide, metolazone, amiloride hydrochloride, spironolactone, triamterene, furosemide, and bumetanide,
beta blocker drug selected from a group comprising acebutolol, atenolol, betaxolol, bisoprolol fumarate, carteolol hydrochloride, metoprolol tartrate, metoprolol succinate, nadolol, penbutolol sulfate, pindolol, propranolol hydrochloride, solotol hydrochloride, and timolol maleate,
a ACE inhibitor drug selected from a group comprising benazepril hydrochloride, captopril, enalapril maleate, fosinopril sodium, lisinopril, moexipril, perindopril, quinapril hydrochloride, ramipril, trandolapril,
angiotensin II receptor blocker selected from a group comprising candesartan, eprosartan mesylate, irbesartan, losartan potassium, telmisartan and valsartan
a calcium channel blocker selected from a group comprising amlodipine besylate, bepridil, diltiazem hydrochloride, felodipine, isradipine, nicardipine, nifedipine, nisoldipine, verapamil and hydrochloride,
an alpha blocker selected from a group comprising doxazosin mesylate, prazosin hydrochloride and terazosin hydrochloride,
an alpha-2 receptor agonist, such as methyldopa,
statin selected from a group comprising atorvastatin, fluvastatin, lovastatin, pravastatin, simvastatin and pitavastatin,
a PCSK9 inhibitor selected from a group comprising evolocumab and alirocumab,
chemotherapic drugs selected from a group comprising 5-fluorouracil, 6-mercaptopurine, cytarabine, gemcitabine, and methotrexate, paclitaxel and rapamycin,
an immunotherapeutic drug selected from a group comprising ipilimumab, nivolumab, pembrolizumab, atezolizumab, avelumab and durvalumab,
genetic material selected from a group comprising single stranded DNA, double stranded DNA, plasmid DNA, siRNA, shRNA, gRNA, sgRNA, tRNA and mRNA,
a pesticide selected from a group comprising herbicide, insecticides, nematicide, molluscicide, piscicide, avicide, rodenticide, bactericide, insect repellent, animal repellent, antimicrobial, and fungicide.

12. A method of making the cyclic polysaccharide compound according to claim 1, comprising the steps of
a) providing a first solution comprising said cyclic polysaccharide compound and a catalyst;
b) adding a monomer of the radical R as defined in any one of claims 1 to 5, to the first solution, thereby providing a reaction mixture;
c) stirring or mixing the reaction mixture at a temperature for a period of time; and
d) isolating the cyclic oligosaccharide or cyclic polysaccharide compound.

13. A method of making the particles according to claim 6, comprising the steps of
e) providing a second solution comprising the cyclic polysaccharide compound according to claim 1, the one or more agent, and a second solvent;
f) providing a third solution comprising the surfactant and a third solvent;
g) contacting the second solution with the third solution, wherein
the second solution contacts the third solution in a microfluidic reactor,
the second solution contacts the third solution under ultrasonic treatment, or
the second solution contacts the third solution by mechanical stirring; and
h) removing the second solvent and the third solvent by evaporation or dialysis.

14. A cosmetic, pharmaceutical, or nutraceutical composition comprising the particles according to claim 6, together with a pharmaceutically, cosmetically, or nutraceutically acceptable carrier.

15. A method of preventing and/or treating a disease in a mammal comprising administering to the mammal the cosmetic, pharmaceutical, or nutraceutical composition according to claim 14.

16. A method of preventing and/or treating a skin disease in a mammal comprising administering to the mammal the cosmetic, pharmaceutical, or nutraceutical composition according to claim 14.

17. A method of supplementing a food, plant, agriculture or ground comprising introducing to the food, plant, agriculture or ground the cosmetic, pharmaceutical, or nutraceutical composition according to claim 14.

18. A method of providing a blood substitute comprising introducing the cosmetic, pharmaceutical, or nutraceutical composition according to claim 14 to a composition.
